SEQUENCE LISTING (24 sequences)

SEQ ID NO: 1 - protein, 9 aa, Homo sapiens
SLRGSLSRL

SEQ ID NO: 2 - protein, 9 aa, Homo sapiens
LLQTRVHSI

SEQ ID NO: 3 - protein, 9 aa, Homo sapiens
ILKKSVHSV

SEQ ID NO: 4 - protein, 9 aa, Homo sapiens
GLMKRLSSL

SEQ ID NO: 5 - protein, 9 aa, Homo sapiens
NLIVNPRSV

SEQ ID NO: 6 - protein, 9 aa, Homo sapiens
LANQELAEV

SEQ ID NO: 7 - protein, 9 aa, Homo sapiens
VTLGGDHSL

SEQ ID NO: 8 - protein, 10 aa, Homo sapiens
FLLRELQDKV

SEQ ID NO: 9 - protein, 9 aa, Homo sapiens
LLRELQDKV

SEQ ID NO: 10 - protein, 9 aa, Homo sapiens
ELQDKVPQL

SEQ ID NO: 11 - protein, 9 aa, Homo sapiens
IVYIGLRDV

SEQ ID NO: 12 - protein, 9 aa, Homo sapiens
KVMERTFDL

SEQ ID NO: 13 - protein, 9 aa, Homo sapiens
YIAEEIHNT

SEQ ID NO: 14 - protein, 9 aa, Homo sapiens
GLLSALDLV

SEQ ID NO: 15 - protein, 10 aa, Homo sapiens
LLSALDLVEV

SEQ ID NO: 16 - protein, 50 aa, Homo sapiens
GFSWIKPCISSASIVYIGLRDVDPPEHFILKNYDIQYFSMRDIDRLGIQK

SEQ ID NO: 17 - protein, 33 aa, Homo sapiens
SLRGSLSRLLQTRVHSILKKSVHSVAVIGAPFS

SEQ ID NO: 18 - protein, 9 aa, Homo sapiens
QIVKNPRSV

SEQ ID NO: 19 - protein, 354 aa, Homo sapiens
MSLRGSLSRLLQTRVHSILKKSVHSVAVIGAPFSQGQKRKGVEHGPAAIREAGLMKRLSSLGCHLKDFGDLSFTPVPKDDLYNNLIVNPRSVGLANQELAEVVSRAVSDGYSCVTLGGDHSLAIGTISGHARHCPDLCVVWVDAHADINTPLTTSSGNLHGQPVSFLLRELQDKVPQLPGFSWIKPCISSASIVYIGLRDVDPPEHFILKNYDIQYFSMRDIDRLGIQKVMERTFDLLIGKRQRPIHLSFDIDAFDPTLAPATGTPVVGGLTYREGMYIAEEIHNTGLLSALDLVEVNPQLATSEEEAKTTANLAVDVIASSFGQTREGGHIVYDQLPTPSSPDESENQARVRI

SEQ ID NO: 20 - protein, 322 aa, Homo sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 21 - protein, 9 aa, Mus musculus
LSPPNIVYI

SEQ ID NO: 22 - protein, 9 aa, Mus musculus
ISAKDIVYI

SEQ ID NO: 23 - protein, 29 aa, Mus musculus
FSWVTPCISAKDIVYIGLRDVDPGEHYII

SEQ ID NO: 24 - protein, 29 aa, Mus musculus
FSWIKPCLSPPNIVYIGLRDVEPPEHFIL

(19) United States
(12) Patent Application Publication
(10) Pub. No.: US 2025/0161422 A1
(43) Pub. Date: May 22, 2025

(54) ARGINASE 2 VACCINE

(71) Applicant: IO Biotech ApS, Copenhagen N (DK)

(72) Inventor: Mads Hald ANDERSEN, Naerum (DK)

(21) Appl. No.: 18/841,338

(22) PCT Filed: Feb. 23, 2023
(86) PCT No.: PCT/EP2023/054568

§ 371 Date: Aug. 23, 2024

(30) Foreign Application Priority Data
Feb. 24, 2022 (GB) 2202547.2
Sep. 13, 2022 (GB) 2213372.2

(51) Int. Cl. A61K 39/00; A61K 39/395; A61P 37/04; C12N 9/78

(52) U.S. Cl. CPC A61K 39/001154; A61K 39/3955; A61P 37/04; C12N 9/78; C12Y 305/03001; A61K 2039/55566

(57) ABSTRACT

The present invention relates to novel polypeptides derived from Arginase 2 (ARG2), polynucleotides encoding said polypeptides, and compositions comprising said polypeptides or polynucleotides. The invention also concerns uses of said polypeptides, polynucleotides and compositions.

### FIELD OF THE INVENTION

The present invention relates to novel polypeptides derived from Arginase 2 (ARG2), polynucleotides encoding said polypeptides, and compositions comprising said polypeptides or polynucleotides. The invention also concerns uses of said polypeptides, polynucleotides and compositions.

### BACKGROUND OF THE INVENTION

Arginases are enzymes that catalyse a reaction which converts the amino acid L-arginine into L-ornithine and urea. This depletes the microenvironment of arginine and leads to a suppression of tumor-specific cytotoxic T-cell responses. Increased Arginase activity has been detected in the cancer cells of patients with, for example, breast, lung, colon or prostate cancer. It has been shown both in vitro and in vivo that mouse macrophages transfected with a rat Arginase gene promote the proliferation of co-cultured tumour cells. Furthermore induction of Arginase expression by macrophages has been shown to increase tumour vascularization through polyamine synthesis. The results of a murine lung carcinoma model showed that there existed a subpopulation of mature tumor-associated myeloid cells that expressed high levels of Arginase. These tumor-associated myeloid cells depleted the extracellular L-Arginine which inhibited antigen-specific proliferation of the tumor infiltrating lymphocytes (TILs). Injection of an Arginase inhibitor blocked the growth of the lung carcinoma in the mice. This shows how induction of Arginase expression in tumor cells and tumor associated myeoloid cells might promote tumor growth by suppression of the anti-tumor immune responses through negative effects on TILs.

MDSCs (myeloid-derived suppressor cells) inhibit the activation, proliferation, and cytotoxicity of effector T cells and natural killer cells, as well as induce Treg differentiation and expansion. Both cancer cells and MDSCs can suppress T cells by manipulating L-arginine metabolism via the enzymes nitric-oxide synthase (NOS) and arginase. Many tumours exhibit increased expressions of arginase and inducible NOS (iNOS), leading to arginine depletion from the tumour microenvironment. Several studies emphasize the importance of this altered tumour arginine metabolism in the suppression of tumour-specific T-cell responses, and it was recently demonstrated that Acute Myeloid Leukemia (AML) blasts show an arginase-dependent ability to inhibit T-cell proliferation and hematopoietic stem cells. Furthermore, arginase and iNOS inhibitors reduce the suppressive activity of AML.

In mammals, two arginase isoenzymes exist: Arginase 1 and Arginase 2. The two isoenzymes catalyse the same biochemical reaction (and thus cannot be disntinguished by enzymatic assays) but differ in cellular expression, regulation and subcellular localisation.

### SUMMARY OF THE INVENTION

The present inventors have previously identified a 50 amino acid region of Arginase 1 (ARG1) and Arginase 2 (ARG2) which is a “hot spot” for immunogenicity. This region corresponds to positions 161-210 of full length human ARG1 (SEQ ID NO: 20) or positions 180-229 of full length human ARG2 (SEQ ID NO: 19), or corresponding positions in murine Arginases. The region and peptides derived from it are described in WO 2018/065563.

The present inventors have also identified that a specific sub-set of polypeptides derived from the “hot spot” region of ARG1 are particularly effective at stimulating immune responses. These peptides correspond to positions 169-206 of full length human ARG1, positions 169-200 of full length human ARGlor positions 169-210 of full length human ARG1 (or corresponding positions in human ARG2 or murine ARG1). This sub-set of polypeptides is described in WO 2020/064744.

The present inventors have further identified that polypeptides derived from an entirely different region of human ARG2 are particularly effective at stimulating immune responses. This region spans the C-terminus of the transit peptide of human ARG2 (position 22 of SEQ ID NO: 19). These peptides are described in WO 2020/099582, including an immunogenic peptide designated A2L2 and corresponds to positions 2-34 of human ARG2 (SEQ ID NO: 16).

The present inventors have now identified further immunogenic polypeptides from ARG2. Unexpectedly, the polypeptides of the present invention are not located in either the “hot spot” region (i.e. amino acids 180-229 of human ARG2) or the A2L2 region (i.e. amino acids 2-34 of ARG2). The polypeptides of the present invention can be advantageously used to stimulate CD8⁺ or CD4⁺ T cells, including cytotoxic CD8⁺ T cells capable of lysing cancer cells (e.g. melanoma cells).

Notably, all previously observed immunogenic responses to immunogenic ARG2 polypeptides have been CD4⁺ T cell responses. However, simultaneous activation of CD4⁺ and CD8⁺ epitopes has been observed to result in strong synergistic protection against tumors. Polypeptides of the invention capable of stimulating CD8⁺ T cells, such as cytotoxic CD8⁺ T cells, are therefore particularly advantageous.

It has also been demonstrated that ARG2-specific T cells generated using the polypeptides of the invention can specifically recognize and react to activated regulatory T cells (Tregs) with high ARG2 expression. The polypeptides of the invention thus have utility in modulating an immunosuppressive tumor microenvironment.

The present invention provides a polypeptide which is an immunogenic fragment of human Arginase 2 (ARG2; SEQ ID NO: 19) that comprises or consists of a sequence of 9-19 consecutive amino acids of SEQ ID NO: 19 provided that said fragment does not comprise the sequence of any one of the following:
amino acids 2-34 of SEQ ID NO: 19;
amino acids 180-229 of SEQ ID NO: 19;
amino acids 129-141 of SEQ ID NO: 19;
amino acids 144-156 of SEQ ID NO: 19; or
amino acids 237-249 of SEQ ID NO: 19.

The polypeptide may comprises or consists of a human leukocyte antigen (HLA) class I restricted epitope. The polypeptide may comprises or consists of a HLA-B8 restricted epitope. The polypeptide may be capable of stimulating CD8⁺ T cells. The CD8⁺ positive T cells may cytotoxic T cells. The CD8⁺ T cells may be ARG2-specific. The CD8⁺ positive T cells may be ARG2-specific cytotoxic T cells. The polypeptide may comprise or consist of the amino acid sequence: NLIVNPRSV (SEQ ID NO: 5).

The polypeptide may comprise or consist of a HLA class II restricted epitope. The polypeptide may be capable of stimulating CD4⁺ T cells. The polypeptide may comprises or consists of the amino acid sequence: GLLSALDLV (SEQ ID NO: 14).

The polypeptide may have a maximum length of 9, 10, 11, 12, 13, 14, 15, 16, 17, 18 or 19 amino acids. The C terminal amino acid of the polypeptide may be replaced with the corresponding amide.

The present invention also provides a polynucleotide encoding a polypeptide of the invention. The polynucleotide may be comprised within a vector.

The present invention further provides a composition comprising a polypeptide of the invention and/or a polynucleotide of the invention. The composition may comprise at least one different polypeptide of the invention and/or at least one different polynucleotide of the invention. The composition may comprise at least one pharmaceutically acceptable diluent, carrier or preservative. The composition may comprise an adjuvant. The adjuvant may be selected from the group consisting of bacterial DNA based adjuvants, oil/surfactant based adjuvants, viral dsRNA based adjuvants, imidazoquinolines, and a Montanide ISA adjuvant.

Also provided by the present invention is a method of treating or preventing a disease or condition in a subject, the method comprising administering to the subject a polypeptide of the invention, a polynucleotide of the invention and/or a composition of the invention.

The present invention also provides a polypeptide of the invention, a polynucleotide of the invention and/or a composition of the invention for use in a method of treating or preventing a disease or condition in a subject.

The present invention further provides the use of a polypeptide of the invention, a polynucleotide of the invention and/or a composition of the invention for the preparation of a medicament for the treatment or prevention of a disease or condition in a subject.

The disease or condition may be characterized at least in part by inappropriate or excessive immune suppressive function of ARG2. The excessive immune suppressive function of ARG2 may be at least in part mediated by activated Treg cells expressing ARG2. The excessive immune suppressive function of ARG2 may be at least in part medicated by cancer-associated fibroblasts expressing ARG2. The disease or condition may be a cancer. The cancer may be a melanoma, (such as a malignant metastatic melanoma), chronic myeloid leukemia (CML), or pancreatic cancer.

Where the disease or condition to be treated is a cancer, the polypeptide of the invention, the polynucleotide of the invention and/or the composition of the invention may be used in combination with an additional cancer therapy. The additional cancer therapy may be an immune system checkpoint inhibitor. Preferably, the immune system checkpoint inhibitor is an antibody. More preferably, the immune system checkpoint inhibitor is an anti-PD1 antibody.

The present invention thus provides a method of treating or preventing a cancer in a subject, the method comprising the administering to the subject a polypeptide of the invention, a polynucleotide of the invention and/or a composition of the invention, wherein the method further comprises the simultaneous or sequential administration of an additional cancer therapy to the subject.

The present invention also provides a polypeptide of the invention, a polynucleotide of the invention and/or a composition of the invention for use in a method of treating or preventing a cancer in a subject, the method comprising the administering to the subject a polypeptide of the invention, a polynucleotide of the invention and/or a composition of the invention, wherein the method further comprises the simultaneous or sequential administration of an additional cancer therapy to the subject.

The present invention further provides the use of a polypeptide of the invention, a polynucleotide of the invention and/or a composition of the invention for the preparation of a medicament for the treatment or prevention of a cancer in a subject wherein the polypeptide, polynucleotide and/or composition is to be administered to the subject simultaneously or sequentially together with an additional cancer therapy.

Further provided by the present invention is a method of stimulating ARG2-specific T cells, the method comprising contacting the cells with a polypeptide of the invention or a composition of the invention. The cells to be contacted may be present in a sample taken from a healthy subject or from a cancer patient. The cells to be contacted may be present in tumor sample. The ARG2-specific T cells may be CD8⁺ positive T cells. Preferably, the ARG2-specific T cells are cytotoxic CD8⁺ T cells.

### BRIEF DESCRIPTION OF THE FIGURES

FIG. 1 shows the identification of ARG2 peptides that elicit CD4+ or CD8⁺ T-cell responses. (A, left) IFNγ ELISPOT screening of responses to 15 different HLA-A2 predicted ARG2-derived peptides in five healthy HLA-A2⁺ donors. 3×10⁵ cells were plated per well. Control and peptide stimulations were carried out in triplicate. Peptide-specific IFNγ secreting cells are given as the difference of spots between cells in the peptide-stimulated wells and control wells. (A, right) Representative examples of the ELISPOT responses shown in (A). (B and C) Representative intracellular cytokine staining for IFNγ and TNFα secreting in samples from three healthy donors stimulated with control or peptide A2S14 (B) or peptide A2S05 (C). Gating strategy is available in FIG. 10. HD=healthy donor. DP=Double positive (TNFα⁺ IFNγ⁺).

FIG. 2 shows that peptide A2S05 elicits responses in both HLA-A2+ and HLA-A2-donors and that responses are also detectable ex vivo. (A) IFNγ ELISPOT responses to A2L2 and A2S05 in 17 healthy donors. 3.5×10⁵ cells were plated per well. Control and peptide stimulations were done in triplicates. Peptide-specific IFNγ secreting cells are given as the difference of spots between cells in the peptide-stimulated wells and control wells. Each dot represents one donor and bars represent the mean. (B) IFNγ ELISPOT responses to A2S05 in 30 healthy donors and 13 cancer patients (two prostate cancer patients, 11 melanoma cancer patients) with known HLA-types. Each dot represents one donor and bars represent the mean. (C) IFNγ ELISPOT responses to two HLA-A2+ and two HLA-A2″ donors. HD=healthy donor. (D) HLA-typing data from the three healthy donors and one cancer patient (AA01, melanoma) with strong responses to A2S05 depicted in (C). HLA-types shared between donors are highlighted in yellow. (E, left) Ex vivo ELISPOT responses to A2S05. 9×10⁵ cells were plated per well and control and peptide stimulations were done in 3-6 replicates. * p≤0.05 (according to the distribution free resampling rule) (E, right) Representative examples of the ex vivo ELISPOT responses.

FIG. 3 shows that ARG2-specific CD8⁺ T cells recognize A2S05 in the context of HLA-B8. (A) ARG2-specific CD8⁺ T cells were expanded from three healthy donors (HD) and one cancer patient (AA01, melanoma). The specificity of the specific T-cell culture was assessed by intracellular cytokine staining for TNFα and IFNγ. CD107a was included as a marker of cytotoxicity. Bars show the percentage of CD8⁺ T cells expressing CD107a and producing IFNγ, TNFα or both (DP, double positive) in response to control stimulation or A2S05 stimulation. (B-D) To identify the HLA-restriction of the ARG2-specific T-cell culture, ARG2-specific T cells from two donors (HD78 and HD93) were evaluated in IFNγ ELISPOT with cancer cells lines pre-pulsed with A2S05 peptide. The same cell lines without peptide stimulation was included as controls. 3×10⁴ ARG2 specific T cells were plated with 1×10⁴ cancer cells (effector to target ratio of 3 to 1). The cell lines were either HLA-A1+ (B), HLA-C7+ (C) or HLA-B8+ (D). T cells plated alone (-) or T cells plated with A2S05 (+pep) served as negative and positive controls, respectively. (E) ⁵¹Cr-release assay showing lysis of FM6 and FM6 stimulated with IFNγ for 24 hrs prior to the assay.

FIG. 4 shows that ARG2-specific T cells recognize and react to stimulated regulatory T cells (Tregs). (A) IFNγ ELISPOT responses of ARG2-specific T cells to in vitro activated and expanded regulatory T cells (Tregs) or effector T cells (Teffs). 5×10⁵ T cells were plated per well with 5×10³ target cells (effector to target ratio of 10 to 1). Bars show the mean±SD. Background from Tregs and Teffs have been corrected in the bars. All conditions were plated in six replicates. * p<0.05 (according to the distribution free resampling rule). (B-C) RT-qPCR analysis of ARG2 expression (B) or FOXP3 expression (C) in the Tregs and Teffs used for the ELISPOT in (A). ARG2 or FOXP3 expression was normalized to the housekeeping gene POL2RA and presented as fold change vs Teff with bars showing the mean±SD of technical triplicates. (D) RT-qPCR analysis of ARG2 expression in Tregs, Teffs and the bulk culture of activated cells from which the subsets were isolated. ARG2 expression is normalized to POL2RA and presented as relative expression (arbitrary units). Bars show the mean±SD of technical triplicates.

FIG. 5 shows ELISPOT responses to ARG2-derived peptides. IFNγ ELISPOT responses of three healthy donors to A2S15 (A), A2S14 (B) or A2S05 (C). 3×10⁵ cells were plated pr. well. Control and peptide stimulations were carried out in triplicate. TNTC=Too numerous to count (>500).

FIG. 6 shows the results of experiments to characterize FM6 cells. (A) Flow cytometry analysis of HLA-ABC and HLA-B8 expression in the metastatic malignant melanoma cell line FM6. Bars indicate the percentage of HLA-ABC⁺ or HLA-B8⁺ cells from the live cell population. (B) Mean fluorescence intensity (MFI) of HLA-B8 in the HLA-B8⁺ population of FM6 cells and FM6 cells stimulated with IFNγ (100 U/ml) for 24 hrs. (C) RT-qPCR analysis of ARG2 expression in FM6. Data is represented as relative expression to the housekeeping gene POL2RA (arbitrary units) with bars showing the mean±SD of technical triplicates.

FIG. 7 shows ELISPOT responses of A2S05-specific T cells to HLA-B8+ cells lines. (A) ARG2-specific T cells from two donors (HD81 and AA01) were evaluated by IFNγ ELISPOT with the metastatic malignant melanoma cell line FM6 pre-pulsed with A2S05 peptide (FM6 pep). FM6 without peptide stimulation (FM6) was included as control. 3×10⁴ ARG2-specific T cells were plated with 1×10⁴ cancer cells (effector to target ratio of 3 to 1). All conditions were set up in triplicates. Bars represent mean±SD. TNTC=Too numerous to count (>500) (B) ARG2-specific T cells from HD93 were evaluated in IFNγ ELISPOT as described above. FM6 cells were pre-pulsed with ARG1-derived peptide (FM6 pep (ARG1)) or A2S05 peptide (FM6 pep (ARG2)). * p<0.05 (according to the distribution free resampling rule). (C) Validation of HLA-B8 restriction of A2S05 by IFNγ ELISPOT of four A2S05-specific T-cell cultures to the three additional HLA-B8+ cell lines (two metastatic malignant melanoma cell lines (FM28 and FM82), and one AML cell line (OCI-M2)). Experiment was performed as described in (A).

FIG. 8 shows the results of sorting and characterization of regulatory T cells (Tregs) and effector T cells (Teff). (A) Flow cytometry plots showing the sorting strategy of Tregs and Teffs in the three donors. Tregs were sorted as CD25^{high}CD127⁻ and Teff were sorted as CD25^{low}CD127⁺. Full gating strategy for the sorting is shown in FIG. 9. (B) Mean fluorescence intensity (MFI) of FOXP3-PE in sorted Tregs and Teffs from the three donors. (C-D) RT-qPCR analysis of IL-10 expression (C) or CTLA4 expression (D) in the Tregs and Teffs. IL-10 or CTLA4 expression was normalized to the housekeeping gene POL2RA and presented as fold change vs Teff with bars showing the mean±SD of technical triplicates. (E) Representative example of RT-qPCR analysis of ARG1 and ARG2 expression in Tregs and Teffs isolated from HD93. Data are represented as relative expression to the housekeeping gene POL2RA (arbitrary units) with bars depicting the mean±SD of technical triplicates.

FIG. 9 shows the Gating strategy for the sorting of regulatory T cells (Treg) or effector T cells (Teff). (A) Gating strategy for the sorting of Tregs or Teffs. Cells were initially gated as Lymphocytes>Singlets>Live cells>CD3+>CD4+. A fluorescence minus one (FMO) control for CD127 was used to set the gate to differentiate CD127+ cells from CD127-cells. Based on this, Tregs were sorted as CD25^{high}CD127 and Teffs were sorted as CD251WCD127+. Treg and Teff gates were placed to aim for sorting of approximately 5% of CD4⁺ T cells. (B) CD127⁻ FITC FMO that was used to set the gate for CD127⁺. (C) FOXP3-PE FMO that was used to set the gate for FOXP3⁺. (D-E) Representative example of purity assessment of sorting quality of Tregs (D) and Teffs (E).

FIG. 10 shows the gating strategy for intracellular cytokine staining for IFNγ and TNFα. Gating strategy for intracellular cytokine staining for IFNγ and TNFα. Cells were initially gated as Lymphocytes>Singlets>Live cells>CD3⁺ and then gated as CD4⁺ or CD8⁺. CD107a expression and IFNγ and TNFα secretion was assessed for CD4⁺ (right) and CD8⁺ (left) T cells separately. A sample without peptide stimulation (Control) was used to set the gates for IFNγ⁺, TNFα⁺ and CD107a⁺.

FIG. 11 shows that ARG2-specific T cells recognize and react to stimulated regulatory T cells (Tregs). (A-C) IFNγ ELISPOT responses of ARG2-specific CD8⁺ T cells to in vitro activated and expanded regulatory T cells (Tregs) or resting CD4⁺ T cells (Trest). 5×10⁴ ARG2-specific CD8⁺ T cells were plated per well with 5×10³ target cells (effector to target ratio of 10 to 1). Bars show the mean IFNγ spot count±SD. Background from Tregs and Trest have been corrected in the bars. All conditions were plated in six replicates. * p<0.05 (according to the distribution free resampling rule). (D) Flow cytometry assessment of the Treg population after co-culture of activated PBMCs with autologous CD8⁺ T cells (Control T cells) or ARG2-specific T cells for 6 hrs. Co-cultures were set up with a 3 to 2 ratio of activated PBMCs to T cells. Activated PBMCs alone was used to set the gates for Tregs, which are similar to the gates used for sorting Tregs for the ELISPOT experiment in (C). Bars show the mean decrease in Treg population when co-cultured with control T cells or ARG2-specific T cells compared to PBMCs plated alone±SD of three technical replicates (depicted as dots). * p≤0.0194 (unpaired t test). (E) RT-qPCR analysis of ARG2 expression in the Tregs and Trest used for the ELISPOT in (A-C). The bulk culture of activated PBMCs from which the Treg and Trest were isolated was also included. ARG2 expression was normalized to the housekeeping gene POL2RA and presented as fold change vs Trest. Bars show the mean±SD of technical triplicates. (F) Western Blot analysis assessing ARG2 expression in sorted Tregs and Trest from HD93. Cell lines Set2 and UKE-1 are included as ARG2 positive and negative controls, respectively. (G-H) IFNγ ELISPOT responses of ARG2-specific CD4⁺ T cells to in vitro activated and expanded Tregs or Trest. 5×10⁴ ARG2-specific CD4⁺ T cells were plated pr. well with 5×10³ target cells (effector to target ratio of 10 to 1). Bars show the mean IFNγ spot count±SD of six technical replicates. Background from Tregs and Trest have been corrected in the bars. All conditions were plated in six replicates. * p≤0.05 (according to the distribution free resampling rule).

FIG. 12 shows the results of sorting and characterization of regulatory T cells (Tregs) and resting CD4⁺ T cells (Trest). (A) Flow cytometry plots showing the sorting strategy of Tregs and Trest in the four donors. Tregs were sorted as CD25^{high}CD127⁻ and Trest were sorted as CD2510WCD127+. Full gating strategy for the sorting is shown in FIG. 9. (B) Representative example of RT-qPCR analysis of ARG1 and ARG2 expression in Tregs and Trest isolated from HD93. Data are represented as relative expression to the housekeeping gene POL2RA (arbitrary units) with bars depicting the mean±SD of technical triplicates. (C) RT-qPCR analysis of FOXP3 expression in the sort-purified Tregs and Trest. FOXP3 expression was normalized to the housekeeping gene POL2RA and presented as fold change vs Trest. Bars show the mean #SD of technical triplicates. (D) Representative example of RT-qPCR analysis of ARG2 expression in Tregs and Trest isolated from HD93 along with autologous ARG2-specific CD8⁺ T cells that were used as effector cells in FIG. 4C. Data are represented as relative expression to the housekeeping gene POL2RA (arbitrary units) with bars depicting the mean±SD of technical triplicates. (E) Purity analysis of the CD8+ enriched fraction. The percentage of live CD3+ cells before MACS-enrichment of CD8+ (pre-sort) and after enrichment are shown. (F, left) Flow cytometry plots showing the sorting strategy of Tregs and Trest in donor AA01. (F, right) Western Blot analysis of ARG2 expression in sorted Tregs, Trest from AA01. Cell lines Set2 and UKE-1 are included as ARG2-positive and negative controls, respectively.

FIG. 13 shows the characterization of sort-purified Tregs. (A) Mean fluorescence intensity (MFI) of FOXP3-PE in sorted Tregs and Trest from the three donors following intracellular staining. (B-F) RT-qPCR analysis of the expression of IL2RA (B), TNFRSF18 (C), IKZF2 (D), CTLA4 (E), or IL10 (F) in the Tregs and Trest. The expression of each Treg signature gene was normalized to the housekeeping gene POL2RA and presented as fold change vs Trest with bars showing the mean±SD of technical triplicates. (G) RT-qPCR analysis of PDCD1 expression in Tregs and Trest. Data is represented as relative expression to the housekeeping gene POL2RA (arbitrary units) with bars showing the mean±SD of technical triplicates. (H) In vitro Treg suppression assay. (Left) Percentage of undivided CD8⁺ T cells after 5 days of co-culture with sort-purified Tregs (+Treg) or Trest (+Trest). Bars indicate the mean of three technical triplicates (depicted as dots). ** p=0.0069 (unpaired t test). (Middle) Proliferation index of CD8 T cells after 5 days of co-culture with Tregs or Trest. Bars indicate the mean of three technical triplicates (depicted as dots). ** p=0.0022 (unpaired t test). (Right) CFSE staining of CD8⁺ T cells co-cultured with Tregs or Trest for 5 days. The dark gray bar (furthest to the right) depicts the undivided population.

FIG. 14 shows the effect of the ARG2-based immune modulatory vaccine on tumor growth, activation of ARG2-specific CD4+ and CD8⁺ T cells and pro-inflammatory tumor microenvironment. (A) (A) Overview of the experimental timeline. (B) Average Pan02 tumor growth for mice receiving a control (Ctrl) or ARG2-peptide immune modulatory vaccine. Mice (n=10 pr. group) were inoculated and vaccinated according to the experimental overview in A. Data are represented as mean±SEM. **** p<0.0001 (2 way ANOVA test). (C) IFNγ ELISPOT on splenocytes isolated from mice receiving the control (n=3) or ARG2-based (n=6) vaccine from the study in shown in B. Briefly, 8×10⁵ splenocytes were plated pr. well. Control and peptide stimulations were performed in triplicates. Peptide-specific IFNγ secreting cells were quantified as the difference in the number of spots counted between the peptide-stimulated and control wells. Each dot represents one mouse and bars represent the mean±SEM. * p=0.0238 (Mann Whitney test). (D) ARG1 and ARG2-specific IFNγ-secreting cells among splenocytes from Pan02 tumor bearing mice receiving the ARG2-based peptide vaccine were assayed with an IFNγ ELISPOT. In this assay, 8×10⁵ splenocytes were plated pr. well with either ARG1 or ARG2 peptide. Each dot represents one mouse, and bars represent the mean±SEM. * p=0.0022 (Mann Whitney test). (E) ARG2-specific IFNγ-secreting cells present in CD4+ and CD8⁺-sorted T cells isolated from splenocytes of Pan02 tumor bearing mice treated with the ARG2-based peptide vaccine were assayed with an IFNγ ELISPOT assay. In this assay, 2.8×10⁵ T cells were plated together with 6×10⁵ antigen-presenting cells (splenocytes from a naïve mouse) with or without ARG2 peptide. Each dot represents one sample (pooled from two mice), and bars represent the mean±SEM. (F) Randomly selected tumors from Pan02 tumor-bearing mice from the experiment in B were harvested (n=4-6 pr. group) on day 31. Tumor RNA was extracted, and bulk RNAseq performed. Differentially expressed genes (FDR<0.05 and absolute log 2 fold-change>0) in Pan02 tumors from ARG2-vaccinated mice compared to control vaccinated mice were identified and presented in a volcano plot. n=282 upregulated and n=33 downregulated genes (see also FIG. 18). (G) Immune-related biological processes (Gene Ontology analysis) associated with significantly upregulated genes in RNAseq data described in F (see also FIG. 19). (H-J) Bar plots showing the absolute immune infiltration score (arbitrary unit) of all immune populations (H) or the immune score ratio of M1 to M2 macrophages (I) and CD8 T cells to Tregs (J). Immune population scores were generated with ImmuCC algoritm, using bulk tumor RNAseq as described in F. Each dot represents one mouse, and bars represent the mean #SEM.

FIG. 15 shows the effect of the immune modulatory ARG2-based peptide vaccine in murine tumor models. (A) Individual tumor growth for Pan02 tumor-bearing mice shown in FIG. 6B. The average tumor growth for each treatment group is also plotted. One mouse showed complete tumor regression (CR) and is indicated by an arrow. (B) Average Pan02 tumor growth for mice receiving an ARG2-peptide immune modulatory vaccine or left untreated. Mice (n=8-9 pr. group) were inoculated and vaccinated similar to the experimental overview in FIG. 6A. Data are represented as mean±SEM. ** p=0.0010 (2 way ANOVA test). (C) Representative example of the percentual change in body weight compared to day 0 in Pan02 tumor-bearing mice left untreated or receiving the ARG2-based immune modulatory peptide vaccine. Mice (n=8-9 pr. group) were treated according to the experimental overview in FIG. 6A. (D) Protein sequence alignment ARG1 (aa162-190) and ARG2 (aa181-209). The ARG2 peptide used in the study (aa188-196) is highlighted in blue, and the corresponding sequence in ARG1 (aa169-177) is highlighted in brown. ARG1_169-177 served as control for specific recognition in FIG. 6D. (E) C57BL/6 were challenged with 5×10⁵ MC38 cells and received ARG2-based or control vaccine on day 0 and 7 post tumor inoculation. On day 18, mice were sacrificed, and spleen and tumors were recovered. (E, left) Peptide-specific IFNγ secreting cells identified by IFNγ ELISPOT on splenocytes from control (n=2) and ARG2 (n=6) vaccinated mice with MC38 tumors (left). 8×10⁵ splenocytes were plated pr. well. Control and peptide stimulations were performed in triplicates. Peptide-specific IFNγ secreting cells are given as the difference of spots between splenocytes in the peptide-stimulated wells and control wells. Each dot represents one mouse, and bars represent mean±SEM. (E, right) Peptide-specific T-cell responses in CD45⁺-enriched cells from the MC38 tumors of mice treated with control (n=2) or ARG2 (n=2) vaccine. 5×10⁵ CD45+ cells were plated pr. well. Control and peptide stimulations were performed in triplicates. Each dot represents one mouse, and bars represent mean±SEM. (F) C57BL/6 mice were challenged with 5×10⁵ LL2 cells and received control or ARG2-vaccine on day 0 and 5 post tumor inoculation. The study was ended on day 12 and spleens and tumors were recovered. (F, left) Peptide-specific IFNγ secreting cells identified by IFNγ ELISPOT on splenocytes from control (n=8) and ARG2 (n=8) vaccinated mice with LL2 tumors. 8×10⁵ splenocytes were plated pr. well. Control and peptide stimulations were performed in triplicates. Each dot represents one mouse, and bars represent mean±SEM. (F, right) Peptide-specific T-cell responses in CD45⁺-enriched cells from the LL2 tumors of mice treated with control (n=4) or ARG2 (n=3) vaccine. 5×10⁵ CD45+ cells were plated pr. well. Control and peptide stimulations were performed in triplicates. Each dot represents one mouse, and bars represent mean±SEM. (G) C57BL/6 mice were challenged with 5×10⁵ B16-F10 cells and received control or ARG2-vaccine on day 0 and 5 post tumor inoculation. The study was ended on day 12 and spleens and tumors were recovered. (G, left) Peptide-specific IFNγ secreting cells identified by IFNγ ELISPOT on splenocytes from control (n=3) and ARG2 (n=3) vaccinated mice with B16-F10 tumors. 8×10⁵ splenocytes were plated pr. well. Control and peptide stimulations were performed in triplicates. Each dot represents one mouse, and bars represent mean±SEM. (G, right) Peptide-specific T-cell responses in CD45⁺-enriched cells from the B16-F10 tumors of mice treated with control (n=3) or ARG2 (n=3) vaccine. 5×10⁵ CD45⁺ cells were plated pr. well. Control and peptide stimulations were performed in triplicates. Each dot represents one mouse, and bars represent mean±SEM. (H) Gene ontology (GO) analysis for biological processes was performed using the Gene Ontology Resource software (http://geneontology.org/) with genes significantly upregulated in ARG2-treated mice as input. Biological processes enriched were initially classified as immune- or non-immune related biological processes and are represented as percentage of total number of enriched biological processes (see also FIG. 19). (1) Percentage of immune related biological processes associated with a positive anti-tumor response, negative anti-tumor response or immune response to bacteria in enriched in mice receiving the ARG2-based vaccine compared to mice receiving the control vaccine (see also FIG. 19).

FIG. 16 shows the immune populations calculated by the ImmuCC algorithm in Pan02 tumor-bearing mice receiving a control or ARG2-based immune modulatory vaccine. ImmuCC analysis for immune populations by analysis of RNAseq data from bulk tumor RNA of Pan02 tumor-bearing mice receiving a control or ARG2-based immune modulatory vaccine as described in FIG. 6F. The box plot indicate the absolute immune score of CD8⁺ T cells (A), CD4⁺ T cells (B), regulatory T cells (C), natural killer cells (D), dendritic cells (E), B cells (F), macrophages (G), monocytes (H) and granulocytes (I) in Pan02 tumor-bearing mice receiving control or ARG2-based vaccines. Subgroups within the different immune populations are listed in Table 5 and 6.

FIG. 17 shows the results of sorting and characterization of regulatory T cells (Tregs) and resting T cells (Trest). (A) Gating strategy for the sorting of Tregs or Trest. Cells were initially gated as Lymphocytes>Singlets>Live cells>CD3⁺>CD4⁺. A fluorescence minus one (FMO) control for CD127 was used to set the gate to differentiate CD127⁺ cells from CD127 cells. Based on this, Tregs were sorted as CD25^{high}CD127⁻ and Trest were sorted as CD25^{low}CD127+. Treg and Trest gates were placed to aim for sorting of approx. 5% of CD4⁺ T cells. (B) CD127-FITC FMO that was used to set the gate for CD127+. (C) FOXP3-PE FMO that was used to set the gate for FOXP3+. (D-E) Representative example of purity assessment of sorting quality of Tregs (D) and Trest (E). (F-G) Representative examples of FOXP3+ cells in Tregs (F) and Trest (G) in HD93.

FIG. 18 shows the supplementary data regarding gene expression information as displayed in FIG. 14F.

FIG. 19 shows the supplementary data regarding the gene ontology analysis as displayed in FIGS. 14G, 15H and 15I.

### BRIEF DESCRIPTION OF THE SEQUENCES

SEQ ID NOs: 1-15 are each an amino acid sequence of a polypeptide derived from human ARG2.

SEQ ID NOs: 16 is the amino acid sequence of the “hot spot” region of human ARG2.

SEQ ID NO: 17 is the amino acid sequence of an ARG2 peptide designated A2L2

SEQ ID NO: 18 is the amino acid sequence of an ARG1 peptide designated ARG1_65-73.

SEQ ID NO: 19 is the amino acid sequence of full length human ARG2.

SEQ ID NO: 20 is the amino acid sequence of full length human ARG1.

SEQ ID NOs: 21 and 22 are the amino acid sequences of immunogenic murine ARG2-derived and murine ARG1-derived epitopes, respectively.

SEQ ID NO: 23 is a murine ARG1 amino acid sequence comprising SEQ ID NO: 22.

SEQ ID NO: 24 is a murine ARG2 amino acid sequence comprising SEQ ID NO: 21.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a polypeptide” includes “polypeptides”, and the like.

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogs and peptidomimetics.

The terms “patient” and “subject” are used interchangeably and typically refer to a human.

All publications, patents and patent applications cited herein, whether supra or infra, are hereby incorporated by reference in their entirety.

The present inventors have identified further polypeptides from human ARG2 which are particularly immunogenic. Unexpectedly, these polypeptides are not located within regions of human ARG2 previously shown immunogenic. In particular, the polypeptides of the invention are not located within either the “hot spot” region or the A2L2 region of human ARG2.

By “immunogenic” herein it is meant that a polypeptide is capable of eliciting an immune response to the ARG2 protein, preferably when said protein is present in or on cells expressing the ARG2 protein. In other words, the polypeptide may be described as immunogenic to ARG2. The polypeptide may alternatively be described as an immunogenic fragment of ARG2. The immune response is preferably a T cell response, and so the polypeptide may be described as an immunogenic fragment of ARG2 comprising a T cell epitope. The immune response may be detected in at least one individual (or in sample taken from the individual) after administration of the polypeptide to said individual (or said sample).

A polypeptide may be identified as immunogenic using any suitable method, including in vitro methods. For example, a peptide may be identified as immunogenic if it has at least one of the following characteristics:
(i) It is capable of eliciting IFN-γ-producing cells in a PBL population of a healthy subject and/or a cancer patient as determined by an ELISPOT assay, and/or
(ii) It is capable of in situ detection in a sample of tumor tissue of CTLs that are reactive with ARG2; and/or
(iii) It is capable of inducing the in vitro growth of specific T-cells.
   Methods suitable for determining whether a polypeptide is immunogenic are also described in the Examples section below.

The polypeptide of the invention is an immunogenic fragment of human Arginase 2 (ARG2; SEQ ID NO: 19) that comprises or consists of a sequence of 9-19 consecutive amino acids of SEQ ID NO: 19 provided that said fragment does not comprise amino acids 2-34 or 180-229 of SEQ ID NO: 19. The polypeptide may have a maximum length of 9, 10, 11, 12, 13, 14, 15, 16, 17, 18 or 19 amino acids and/or in which the C terminal amino acid is replaced with the corresponding amide. The polypeptide may be isolated.

The polypeptide may comprise or consist of a human leukocyte antigen (HLA) class I restricted epitope. The polypeptide may comprise of consist of a HLA-B8 restricted epitope. The polypeptide may be capable of stimulating CD8⁺ T cells. The CD8⁺ T cells may be cytotoxic T cells. The CD8⁺ T cells may be ARG2-specific. An exemplary polypeptide of this type comprises or consists of the amino acid sequence: NLIVNPRSV (SEQ ID NO: 5; A2S05). Peptide A2S05 is HLA-B8 restricted and capable of stimulating ARG2-specific CD8+ cytotoxic T cells.

The polypeptide may comprises or consists of a HLA class II restricted epitope. The polypeptide may be capable of stimulating CD4⁺ T cells. An exemplary polypeptide of this type may comprise or consist of the of the amino acid sequence: GLLSALDLV (SEQ ID NO: 14).

In any polypeptide described herein, the amino acid sequence may be modified by one, two, three, four, or five (that is up to five) additions, deletions or substitutions, provided that a polypeptide having the modified sequence exhibits the same or increased immunogenicity to ARG2, as compared to a polypeptide having the unmodified sequence. By “the same” it is to be understood that the polypeptide of the modified sequence does not exhibit significantly reduced immunogenicity to ARG2 as compared to polypeptide of the unmodified sequence. Any comparison of immunogenicity between sequences is to be conducted using the same assay. Unless otherwise specified, modifications to a polypeptide sequence are preferably conservative amino acid substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table A1 below. Where amino acids have similar polarity, this can be determined by reference to the hydropathy scale for amino acid side chains in Table A2.

TABLE Al
Chemical properties of amino acids

| | | | |
|---|---|---|---|
| Ala (A) | aliphatic, hydrophobic, | Met (M) | hydrophobic, |
| | neutral | | neutral |
| Cys (C) | polar, hydrophobic, | Asn (N) | polar, hydrophilic, |
| | neutral | | neutral |
| Asp (D) | polar, hydrophilic, | Pro (P) | hydrophobic, |
| | charged (−) | | neutral |
| Glu (E) | polar, hydrophilic, | Gln (Q) | polar, hydrophilic, |
| | charged (−) | | neutral |
| Phe (F) | aromatic, hydrophobic, | Arg (R) | polar, hydrophilic, |
| | neutral | | charged (+) |
| Gly (G) | aliphatic, | Ser (S) | polar, hydrophilic, |
| | neutral | | neutral |
| His (H) | aromatic, polar, | Thr (T) | polar, hydrophilic, |
| | hydrophilic, charged (+) | | neutral |
| Ile (I) | aliphatic, hydrophobic, | Val (V) | aliphatic, hydrophobic, |
| | neutral | | neutral |
| Lys (K) | polar, hydrophilic, | Trp (W) | aromatic, hydrophobic, |
| | charged(+) | | neutral |
| Leu (L) | aliphatic, hydrophobic, | Tyr (Y) | aromatic, polar, |
| | neutral | | hydrophobic |

TABLE A2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

In any polypeptide disclosed herein, any one or more of the following modifications may be made to improve physiochemical properties (e.g. stability), provided that the polypeptide exhibits the same or increased immunogenicity to ARG2, as compared to a polypeptide having the unmodified sequence:
a) Replacement of the C terminal amino acid with the corresponding amide (may increase resistance to carboxypeptidases);
b) Replacement of the N terminal amino acid with the corresponding acylated amino acid (may increase resistance to aminopeptidases);
c) Replacement of one or more amino acids with the corresponding methylated amino acids (may improve proteolytic resistance);
d) Replacement of one or more amino acids with the corresponding amino acid in D-configuration (may improve proteolytic resistance).

Any polypeptide disclosed herein may have attached at the N and/or C terminus at least one additional moiety to improve solubility, stability and/or to aid with manufacture/isolation, provided that the polypeptide exhibits the same or increased immunogenicity to ARG2, as compared to a polypeptide lacking the additional moiety. Suitable moieties include hydrophilic amino acids. For example, the amino acid sequences KK, KR or RR may be added at the N terminus and/or C terminus. Other suitable moieties include Albumin or PEG (Polyethylene Glycol).

A polypeptide as disclosed herein may be produced by any suitable means. For example, the polypeptide may be synthesised directly using standard techniques known in the art, such as Fmoc solid phase chemistry, Boc solid phase chemistry or by solution phase peptide synthesis. Alternatively, a polypeptide may be produced by transforming a cell, typically a bacterial cell, with a nucleic acid molecule or vector which encodes said polypeptide.

The invention provides nucleic acid molecules and vectors which encode a polypeptide of the invention. The invention also provides a host cell comprising such a nucleic acid or vector.

The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers.

Where the polynucleotide of the invention is an mRNA it may be provided as an mRNA vaccine. Said mRNA vaccine may be formulated as a lipid nanoparticle composition, for example a lipid nanoparticle composition comprising an mRNA encoding a polypeptide of the invention and an ionizable lipid.

A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences, for example in an expression vector. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides can be synthesised according to methods well known in the art, as described by way of example in Sambrook et al (1989, Molecular Cloning—a laboratory manual; Cold Spring Harbor Press). The nucleic acid molecules of the present invention may be provided in the form of an expression cassette which includes control sequences operably linked to the inserted sequence, thus allowing for expression of the polypeptide of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al. (1989, Molecular Cloning—a laboratory manual; Cold Spring Harbor Press)

The invention also includes cells that have been modified to express a polypeptide of the invention. Such cells typically include prokaryotic cells such as bacterial cells, for example E. coli. Such cells may be cultured using routine methods to produce a polypeptide of the invention.

The polypeptide of the invention may be in a substantially isolated form. It may be mixed with carriers, preservatives, or diluents (discussed below) which will not interfere with the intended use, and/or with an adjuvant (also discussed below) and still be regarded as substantially isolated. It may also be in a substantially purified form, in which case it will generally comprise at least 90%, e.g. at least 95%, 98% or 99%, of the protein in the preparation.

### Compositions Comprising Polypeptides or Polynucleotides

In another aspect, the present invention provides a composition comprising a polypeptide of the invention and/or a polynucleotide of the invention. For example, example, the invention provides a composition comprising one or more polypeptides of the invention and/or one or more polynucleotides of the invention, and optionally at least one adjuvant, pharmaceutically acceptable carrier, preservative and/or excipient.

The composition may comprise at least two, at least three, at least four, at least five, at least six, at least seven, at least eight different polypeptides of the invention and optionally at least one adjuvant, pharmaceutically acceptable carrier, preservative and/or excipient.

The composition may comprise at least two, at least three, at least four, at least five, at least six, at least seven, at least eight different polynucleotides of the invention and optionally at least one adjuvant, pharmaceutically acceptable carrier, preservative and/or excipient.

The carrier, preservative and excipient must be ‘acceptable’ in the sense of being compatible with the other ingredients of the composition and not deleterious to a subject to which the composition is administered. Typically, all components and the final composition are sterile and pyrogen free. The composition may be a pharmaceutical composition. The composition may preferably comprise an adjuvant.

Adjuvants are any substance whose admixture into the composition increases or otherwise modifies the immune response elicited by the composition. Adjuvants, broadly defined, are substances which promote immune responses. Adjuvants may also preferably have a depot effect, in that they also result in a slow and sustained release of an active agent from the administration site. A general discussion of adjuvants is provided in Goding, Monoclonal Antibodies: Principles & Practice (2nd edition, 1986) at pages 61-63.

Adjuvants may be selected from the group consisting of: AIK(SO₄)₂, AlNa(SO₄)₂, AlNH₄ (SO₄), silica, alum, Al(OH)₃, Ca₃(PO₄)₂, kaolin, carbon, aluminum hydroxide, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′2′-dipalmitoyl-sn-glycero-3-hydroxphosphoryloxy)-ethylamine (CGP 19835A, also referred to as MTP-PE), RIBI (MPL⁺ TDM+CWS) in a 2% squalene/Tween-80.RTM. emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium, tuberculosis, substances found in Corynebacterium parvum, Bordetella pertussis, and members of the genus Brucella, Titermax, ISCOMS, Quil A, ALUN (see US 58767 and 5,554,372), Lipid A derivatives, choleratoxin derivatives, HSP derivatives, LPS derivatives, synthetic peptide matrixes or GMDP, Interleukin 1, Interleukin 2, Montanide ISA-51 and QS-21. Various saponin extracts have also been suggested to be useful as adjuvants in immunogenic compositions. Granulocyte-macrophage colony stimulating factor (GM-CSF) may also be used as an adjuvant.

Preferred adjuvants to be used with the invention include oil/surfactant based adjuvants such as Montanide adjuvants (available from Seppic, Belgium), preferably Montanide ISA-51. Other preferred adjuvants are bacterial DNA based adjuvants, such as adjuvants including CpG oligonucleotide sequences. Yet other preferred adjuvants are viral dsRNA based adjuvants, such as poly I:C. GM-CSF and imidazoquinolines are also examples of preferred adjuvants.

The adjuvant is most preferably a Montanide ISA adjuvant. The Montanide ISA adjuvant is preferably Montanide ISA 51 or Montanide ISA 720.

In Goding, Monoclonal Antibodies: Principles & Practice (2nd edition, 1986) at pages 61-63 it is also noted that, when an antigen of interest is of low molecular weight, or is poorly immunogenic, coupling to an immunogenic carrier is recommended. A polypeptide of the invention may therefore be coupled to a carrier. A carrier may be present independently of an adjuvant. The function of a carrier can be, for example, to increase the molecular weight of a polypeptide fragment in order to increase activity or immunogenicity, to confer stability, to increase the biological activity, or to increase serum half-life. Furthermore, a carrier may aid in presenting the polypeptide or fragment thereof to T-cells. Thus, in the composition, the polypeptide may be associated with a carrier such as those set out below.

The carrier may be any suitable carrier known to a person skilled in the art, for example a protein or an antigen presenting cell, such as a dendritic cell (DC). Carrier proteins include keyhole limpet hemocyanin, serum proteins such as transferrin, bovine serum albumin, human serum albumin, thyroglobulin or ovalbumin, immunoglobulins, or hormones, such as insulin or palmitic acid. Alternatively the carrier protein may be tetanus toxoid or diphtheria toxoid. Alternatively, the carrier may be a dextran such as sepharose. The carrier must be physiologically acceptable to humans and safe.

If the composition comprises an excipient, it must be ‘pharmaceutically acceptable’ in the sense of being compatible with the other ingredients of the composition and not deleterious to the recipient thereof. Auxiliary substances, such as wetting or emulsifying agents, pH buffering substances and the like, may be present in the excipient. These excipients and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

Formulation of a suitable composition can be carried out using standard pharmaceutical formulation chemistries and methodologies all of which are readily available to the reasonably skilled artisan. Such compositions may be prepared, packaged, or sold in a form suitable for bolus administration or for continuous administration. Injectable compositions may be prepared, packaged, or sold in unit dosage form, such as in ampoules or in multi-dose containers optionally containing a preservative. Compositions include, but are not limited to, suspensions, solutions, emulsions in oily or aqueous vehicles, pastes, and implantable sustained-release or biodegradable formulations. In one embodiment of a composition, the active ingredient is provided in dry (for e.g., a powder or granules) form for reconstitution with a suitable vehicle (e.g., sterile pyrogen-free water) prior to administration of the reconstituted composition. The composition may be prepared, packaged, or sold in the form of a sterile injectable aqueous or oily suspension or solution. This suspension or solution may be formulated according to the known art, and may comprise, in addition to the active ingredient, additional ingredients such as the adjuvants, excipients and auxiliary substances described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. Alternatively, the active ingredients of the composition may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

### Methods of Use

The polypeptide, polynucleotide or composition of the invention may be used in a method of treating or preventing a disease or condition in a subject. The polypeptide, polynucleotide, or composition of the invention may be used in the manufacture of a medicament for use in a method of treating or preventing a disease or condition in a subject. The method comprises administering to the said subject the said polypeptide, the said polynucleotide or the said composition. Administration may be of a therapeutically or prophylactically effective quantity of the said polypeptide, the said polynucleotide, or the said composition, to a subject in need thereof.

The disease or condition may be characterized at least in part by inappropriate or excessive immune suppressive function of ARG2. The disease or condition may be a cancer, preferably a cancer which expresses ARG2 and/or which is associated with inappropriate or excessive immune suppressive function of ARG2. The cancer may be a cancer of the kidney, prostate, breast, brain, pancreas, head and neck, or small intestine, or may be a colorectal or gastric cancer, or may be a melanoma, or may be a leukemia, such as acute myeloid leukemia (AML), Chronic lymphocytic leukemia (CLL), or chronic myeloid leukemia (CML). Preferably, the cancer is a melanoma (such as a malignant melanoma), CML, or a pancreatic cancer. The cancer may be CML characterized by inappropriate or excessive immune suppressive function of ARG2. The cancer may be a melanoma characterized by inappropriate or excessive immune suppressive function of ARG2. The cancer may be a malignant melanoma characterized by inappropriate or excessive immune suppressive function of ARG2. The cancer may be a pancreatic cancer. The excessive immune suppressive function of ARG2 may be mediated at least in part by activated Treg cells characterized by expression of ARG2. The excessive immune suppressive function of ARG2 may be mediated at least in part by cancer-associated fibroblasts (CAFs) characterized by expression of ARG2. The cancer may be resistant to other cancer therapies, in particular it may be resistant to to immune system checkpoint inhibitors such as anti-PD1 therapy.

The method may comprise simultaneous or sequential administration with an additional cancer therapy. The additional cancer therapy may be selected from a cytokine therapy, a T-cell therapy, an NK therapy, an immune system checkpoint inhibitor, chemotherapy, radiotherapy, immunostimulating substances, gene therapy, or an antibody.

Immune system checkpoint inhibitors are preferred as an additional cancer therapy. Vaccination against ARG2 may have a synergistic effect when combined with inhibition of an immune system checkpoint. Examples of immune system checkpoints include:
a) The interaction between Indoleamine 2,3-dioxygenase (IDO1) and its substrate;
b) The interaction between PD1 and PDL1 and/or PD1 and PDL2;
c) The interaction between CTLA4 and CD86 and/or CTLA4 and CD80;
d) The interaction between B7-H3 and/or B7-H4 and their respective ligands;
e) The interaction between HVEM and BTLA;
f) The interaction between GAL9 and TIM3;
g) The interaction between MHC class I or II and LAG3; and
h) The interaction between MHC class I or II and KIR.
   Inhibition of checkpoints (a), (b) and (c) is particularly preferred as an additional cancer therapy. A checkpoint inhibitor may be any immunomodulatory agent (such as an antibody) which blocks or inhibits an immune system checkpoint, or it may be an immunotherapeutic composition comprising a component of an immune system checkpoint, or an immunogenic fragment of said component, which stimulates targeting of the checkpoint by the immune system.

The additional cancer therapy may be an antibody.

The antibody may be Abagovomab, Abciximab, Actoxumab, Adalimumab, Adecatumumab, Afelimomab, Afutuzumab, Alacizumab pegol, ALD518, Alemtuzumab, Alirocumab, Altumomab pentetate, Amatuximab, Anatumomab mafenatox, Anrukinzumab, Apolizumab, Arcitumomab, Aselizumab, Atinumab, Atlizumab (=tocilizumab), Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, CC49, Cedelizumab, Certolizumab pegol, Cetuximab, Ch.14.18, Citatuzumab bogatox, Cixutumumab, Clazakizumab, Clenoliximab, Clivatuzumab tetraxetan, Conatumumab, Concizumab, Crenezumab, CR6261, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Elotuzumab Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab Farletuzumab, Fasinumab, FBTA05, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, GS6624, Ibalizumab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Obinutuzumab, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN1412, Ticilimumab (=tremelimumab), Tildrakizumab, Tigatuzumab, TNX-650, Tocilizumab (=atlizumab), Toralizumab, Tositumomab, Tralokinumab, Trastuzumab, TRBS07, Tregalizumab, Tremelimumab Tucotuzumab celmoleukin, Tuvirumab, Ublituximab, Urelumab, Urtoxazumab, Ustekinumab, Vapaliximab, Vatelizumab, Vedolizumab, Veltuzumab, Vepalimomab Vesencumab, Visilizumab, Volociximab, Vorsetuzumab mafodotin, Votumumab, Zalutumumab, Zanolimumab, Zatuximab, Ziralimumab or Zolimomab aritox.

Preferred antibodies include Natalizumab, Vedolizumab, Belimumab, Atacicept, Alefacept, Otelixizumab, Teplizumab, Rituximab, Ofatumumab, Ocrelizumab, Epratuzumab, Alemtuzumab, Abatacept, Eculizumab, Omalizumab, Canakinumab, Meplizumab, Reslizumab, Tocilizumab, Ustekinumab, Briakinumab, Etanercept, Inlfliximab, Adalimumab, Certolizumab pegol, Golimumab, Trastuzumab, Gemtuzumab, Ozogamicin, Ibritumomab, Tiuxetan, Tostitumomab, Cetuximab, Bevacizumab, Panitumumab, Denosumab, Ipilimumab, Brentuximab and Vedotin.

Particularly preferred antibodies that may be used in the method of the invention include: daratumumab, nivolumab, pembrolizumab, avelumab, rituximab, trastuzumab, pertuzumab, alemtuzumab, cetuximab, panitumumab, tositumomab and ofatumumab. Anti-PD1 antibodies such as nivolumab and pembrolizumab are also especially preferred. Pembrolizumab is most preferred.

The additional cancer therapy may be selected from the group consisting of Actimide, Azacitidine, Azathioprine, Bleomycin, Carboplatin, Capecitabine, Cisplatin, Chlorambucil, Cyclophosphamide, Cytarabine, Dauno-rubicin, Docetaxel, Doxifluridine, Doxorubicin, Epirubicin, Etoposide, Fludarabine, Fluor-ouracil, Gemcitabine, Hydroxyurea, Idarubicin, Irinotecan, Lenalidomide, Leucovorin, Mechlorethamine, Melphalan, Mercaptopurine, Methotrexate, Mitoxantrone, Oxaliplatin, Paclitaxel, Pemetrexed, Revlimid, Temozolomide, Teniposide, Thioguanine, Valrubicin, Vinblastine, Vincristine, Vindesine and Vinorelbine.

The polypeptide or composition of the invention may also be used in a method of stimulating ARG2-specific T cells, such as CD4+ and CD8⁺ T-cells, comprising contacting cells with the said polypeptide or composition. The method may be conducted ex vivo. The method may be conducted in vitro. The cells may be present in a sample taken from a healthy subject or from a cancer patient, such as in a tumour sample. The method may be a method of stimulating CD8⁺ T cells. The CD8⁺ T cells may be cytotoxic CD8⁺ T cells. The polypeptides used in such methods of simulating CD8⁺ T cells may comprise or consist of a human leukocyte antigen (HLA) class I restricted epitope, optionally an HLA-B8 restricted epitope. For example, a polypeptide comprising or consisting of the amino acid sequence of NLIVNPRSV (SEQ ID NO: 5) may be used in a method of simulating CD8⁺ T cells, in particular cytotoxic CD8⁺ T cells.

The present invention is further illustrated by the following examples that, however, are not to be construed as limiting the scope of protection. The features disclosed in the foregoing description and in the following examples may, both separately and in any combination thereof, be material for realizing the invention in diverse forms thereof.

### EXAMPLES

### Example 1—Materials and Methods

### Donor Material

Healthy donors PBMCs were isolated using density gradient separation over Lymphoprep™ (Alere) and cryopreserved at −150° C. in fetal bovine serum (FBS, Life Technologies) supplemented with 10% dimethyl sulfide (DMSO). PBMCs from cancer patients with solid tumors were derived from blood samples drawn minimum four weeks after administration of any therapy. PBMCs were maintained in X-Vivo (BioNordika) supplemented with 5% human serum (Sigma Aldrich). All patient protocols were approved by the Scientific Ethics Committee for the Capital Region of Denmark and conducted in accordance with the provisions of the Declaration of Helsinki. Written informed consent was obtained from patients before study entry.

### Peptides

Peptide sequences were predicted using algorithms available at www.syfpeithi.de_and cbs.dtu.dk. The list of short peptides was selected to only include peptide with a score above 24 (SYFPEITHI) or with a rank below 1.5 (NetMHC). All peptides were synthesized by Schäfer and had a purity above 90%. Peptides were dissolved in 100% DMSO to a stock concentration of 10 mM or in sterile water to a concentration of 2 mM. Peptides dissolved in water were sterile filtered before use. All peptides used in this study are listed in TABLE 1.

### In Vitro Stimulation and IFNγ ELISPOT

PBMCs from healthy donors or cancer patients were subjected to in vitro stimulation with ARG2-derived peptides by plating the cells with 10 μM peptide. The following day, the cells received low-dose IL-2 (120 U/ml) treatment. At 10-14 days after peptide stimulation, the cells were used for IFNγ ELISPOT. ELISPOT plates were coated with 7.5 μg/μl IFNγ capture antibody (MabTech) overnight. The next day, the plates were washed and blocked in X-vivo media followed by the plating of 2.5-3.5×10⁵ PBMCs per well for ELISPOT, with and without restimulation with 5 μM peptide. A minimum of triplicate wells were set up pr. condition. The cells were incubated 14-16 hours before they were washed and a biotin-conjugated secondary antibody (Mabtech) was added, followed by a 2-hour incubation.

Next, the cells were washed again, followed by incubation with streptavidin-conjugated alkaline phosphatase (Mabtech) for 1 hour. Finally, spots were developed by addition of BCIP/NBT substrate (Mabtech). Tap water was used to stop the reaction. Spots corresponding to IFNγ secretion were quantified by visualization on a CTL ImmunoSpot S6 Ultimate-V analyzer with ImmunoSpot software version 5.1.

Peptide-specific IFNγ secreting cells were calculated by subtracting the average spot count in control wells from the average spot count in peptide-stimulated wells. ELISPOT assays with ARG2-specific T cells (effector cells) and various immune cells or cancer cells as target cells were set up by adding 3×10⁴-5×10⁴ effector cells to ELISPOT wells followed by 5×10³-10⁴ target cells. Peptide pulsing was performed by incubating the target cells with 20 μM peptide for 1 hour, followed by two washes to remove unbound peptide. Effector cells with added peptide served as the positive control and target cells plated without target cells as the negative control. In ELISPOTs assays with immune cells as target cells, wells containing only effector cells were also included as controls. The average spot count from replicate wells was subtracted from the spot count of effector cells plated with the respective target cells.

### Ex Vivo ELISPOT

PBMCs were thawed and rested overnight. The next day, 9×10⁵ cells were plated pr. well. Control and peptide stimulations were performed in at least triplicate. The rest of the protocol was performed as described above.

### Cell Lines

K562, K562-A1, FM6, FM28, and FM82 cell lines were maintained in RMPI-1640 (Gibco) supplemented with 10% FBS. OCI-M2 was maintained in Iscove's MDM (Gibco) with 20% FBS. Cells were passaged every 2-3 days. Adherent cells (FM6, FM28, and FM82) were passaged following detachment from the flask with 0.25% Trypsin (Gibco). All cells lines were confirmed to be mycoplasma negative.

### Intracellular Cytokine Staining

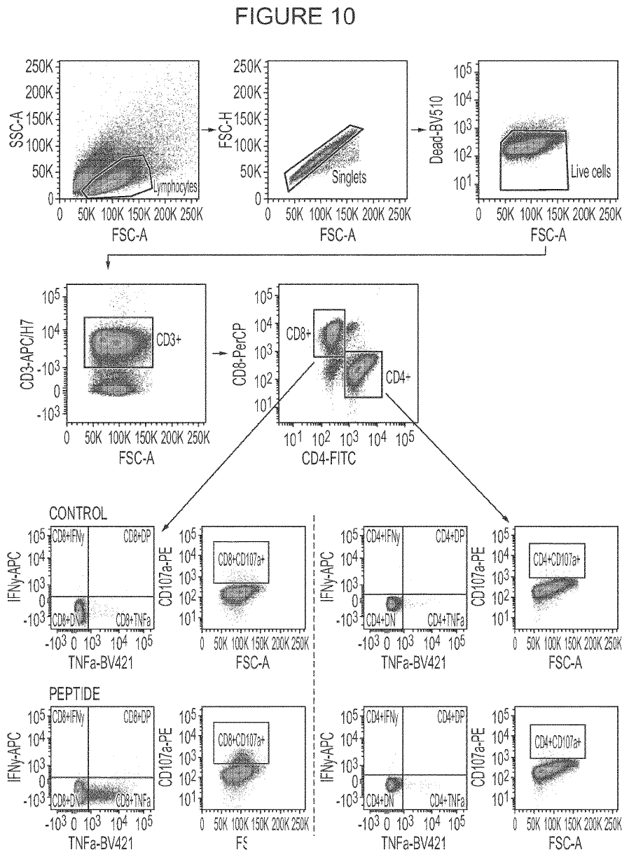

PBMCs were in vitro stimulated with peptide as described above. At 10-14 days post-simulation, the cells were used for intracellular cytokine staining. Cells were incubated with peptide or a no-peptide control and CD107a-PE (BD) for 1 h, followed by addition of GolgiPlug (BD). After a 4-hour incubation period, the cells were washed and stained for the extracellular markers CD3-APC/H7, CD4-FITC, and CD8-PerCP, and dead cells were stained with FVS-510. Next, the cells were permeabilized using Fixation/Permeabilization buffer (Invitrogen), followed by staining with IFNγ-APC and TNFα-BV421. Data were acquired using a BD Canto II flow cytometer and analyzed using FlowJo. The gating strategy is presented in FIG. 10, and the antibodies used in this study are listed in Table 2 below.

TABLE 2
List of antibodies used in this study

| | | CLONE, | |
|---|---|---|---|
| | FLUORO- | CATALOG NUMBER, | |
| TARGET | CHROME | MANUFACTURER | APPLICATION |
| CD3 | APC-H7 | SK7, 641415, BD | ICS |
| CD4 | FITC | SK3, 345768, BD | ICS |
| CD8 | PerCP | SK1, 345774, BD | ICS |
| CD107a | PE | H4A3, 560948, BD | ICS |
| IFNγ | APC | 25723.11, 341117, BD | ICS |
| TNFα | BV421 | MAb11, 562783, BD | ICS |
| Dead cell | BV510 | N/A, 564406, BD | Viability |
| marker* | | | |
| HLA-A02 | PE | BB7.2, 558570, BD | HLA-typing |
| HLA-ABC | FITC | DX17, 560168, BD | HLA-typing |
| HLA-B8 | PE | REA145, 130-119-137, | HLA-typing |
| | | Miltenyi Biotech | |
| CD4 | PerCP | SK3, 566924, BD | Treg isolation |
| CD25 | Pe-Cy7 | M-A251, 557741, BD | Treg isolation |
| CD127 | FITC | HIL-7R-M21, 560549, BD | Treg isolation |
| FOXP3 | PE | 259D/C7, 560046, BD | Treg isolation |
| ARG2 | N/A | EPR9473, ab137069, abcam | Western blot |
| Vinculin | N/A | V284, SAB4200080, | Western blot |
| | | Sigma-Aldrich | |
| Anti-rabbit, | N/A | N/A, CST7074, Cell | Western blot |
| IgG, | | Signaling Technology | |
| HRP-linked | | | |
| Anti-mous, | N/A | N/A, CST7076, Cell | Western blot |
| IgG, | | Signaling Technology | |
| HRP-linked | | | |

*The dead cell marker is Fixable Viability Stain 510 and thus not an antibody.
N/A = not applicable

### Flow Cytometry-Based Analysis of HLA Expression

Pan-HLA (HLA-ABC) and HLA-B8 expression levels in the cell lines were analyzed by staining with HLA-ABC-FITC (BD) and HLA-B8-PE (Miltenyi Biotec), respectively. The HLA-negative cell line K562 was used to set the gates for HLA-ABC+ and HLA-B8+ cells. Data were acquired using a BD Canto II flow cytometer and analyzed using FlowJo. The antibodies used in this study are listed in Table 2 above.

### Generation of ARG2-Specific T-Cell Cultures

ARG2-specific T-cell cultures were obtained by stimulation of PBMCs with ARG2-S05 peptide, followed by low-dose IL-2 (120 U/mL) the next day. At 12 days after peptide stimulation, the cells were re-stimulated with peptide, and IFNγ-secreting cells were isolated using magnetic beads. Next, these cells were expanded using a rapid expansion protocol, including feeder cells, CD3 antibody, and high-dose IL-2 (3000-6000 U/mL). On day 16-17, culture specificity was determined by stimulation with ARG2-S05 peptide and determination of the cytokine release by ICS.

### Activation, Expansion, and Isolation of Regulatory T Cells (Tregs) and Effector T Cells (Teffs) or Resting T Cells (Trest)

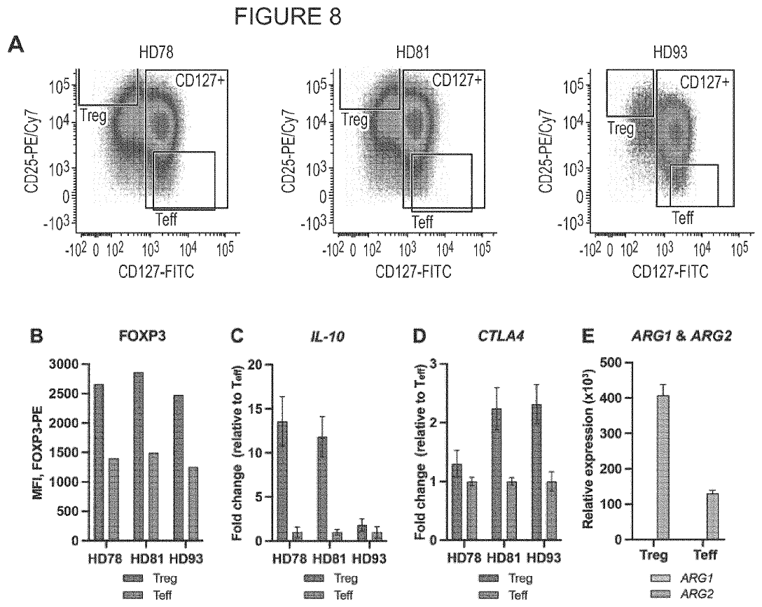

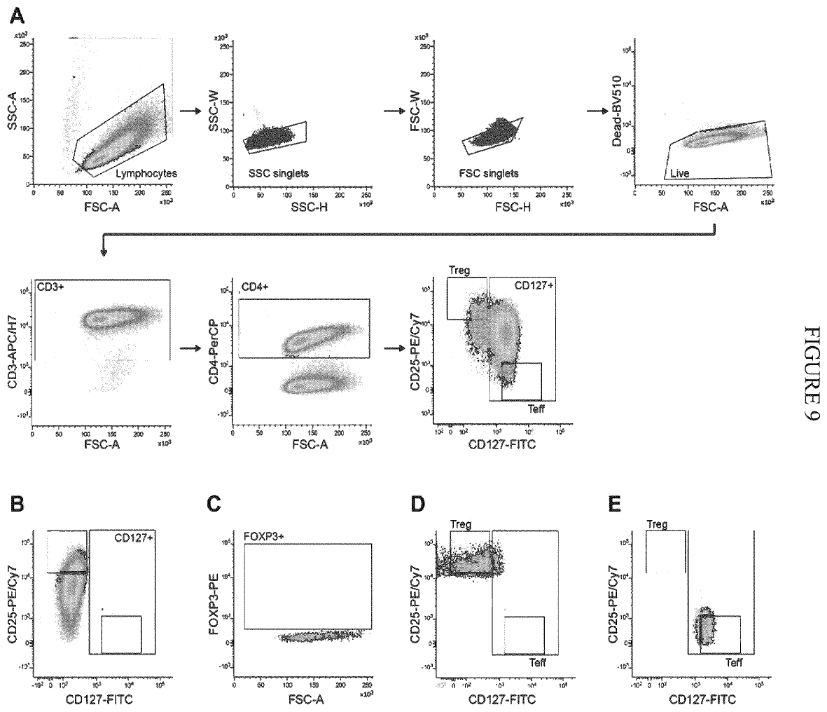

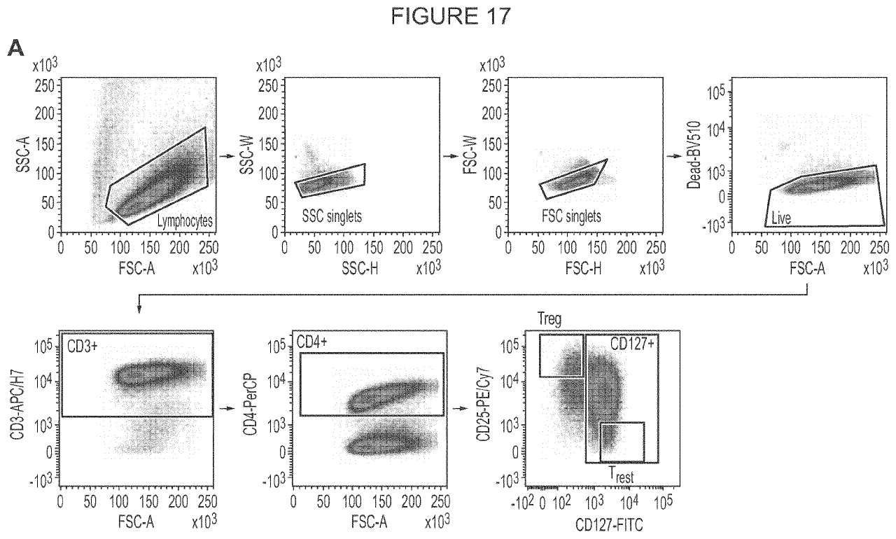

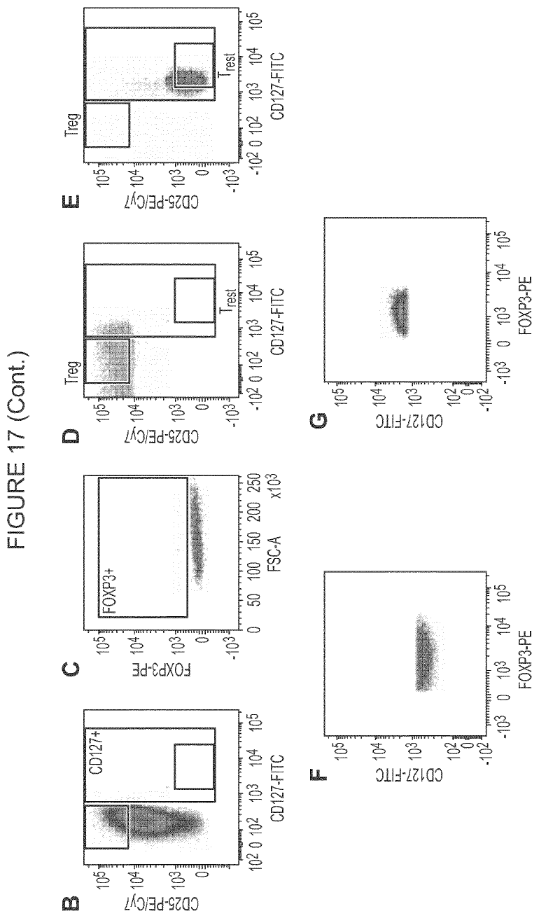

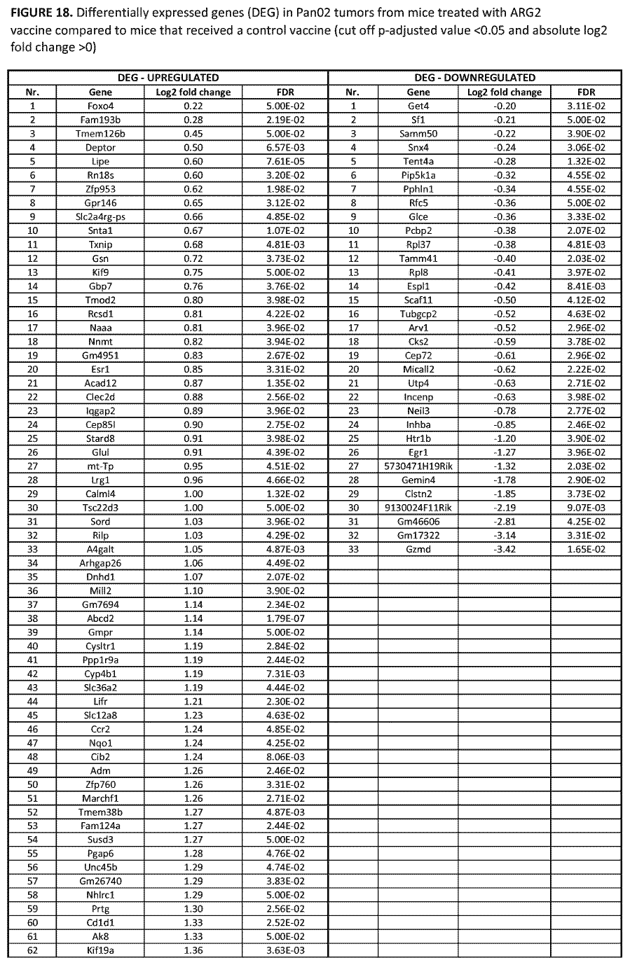

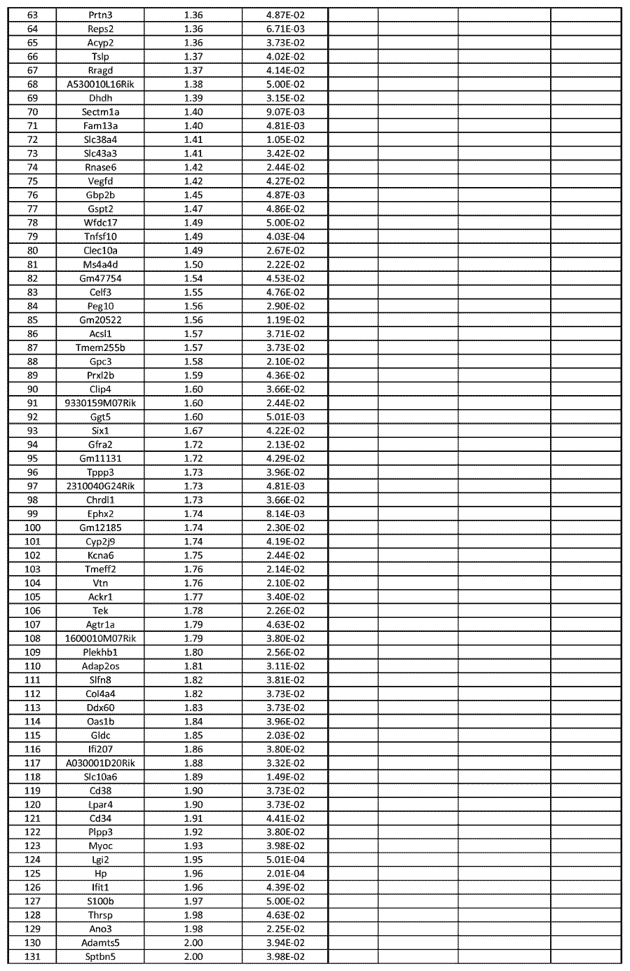

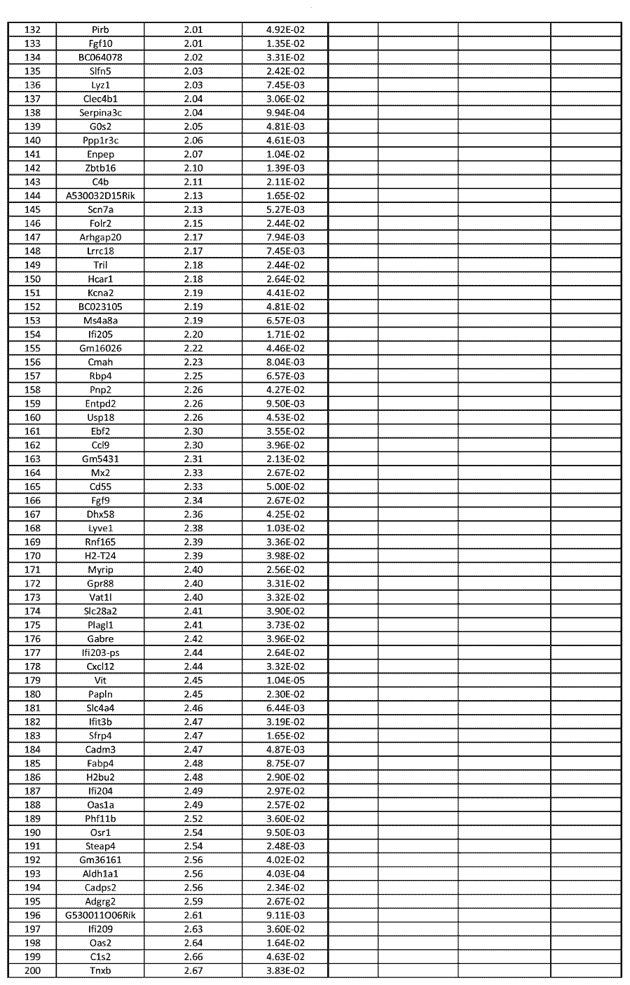

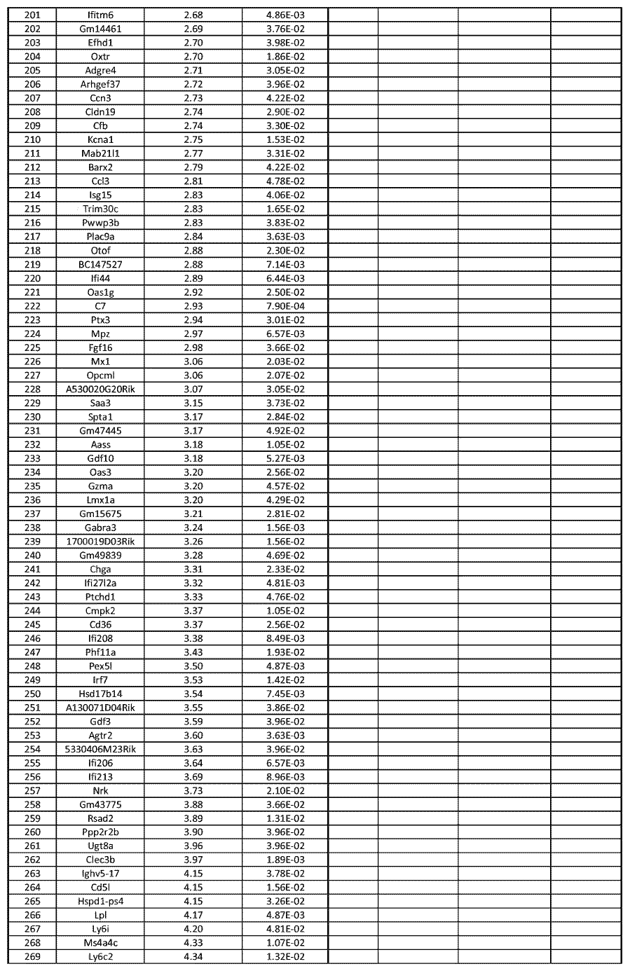

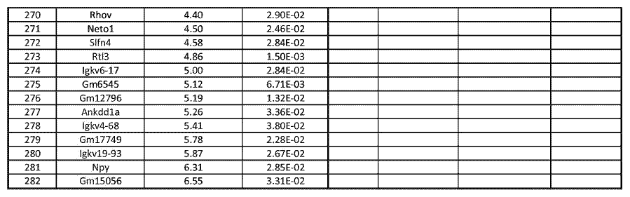

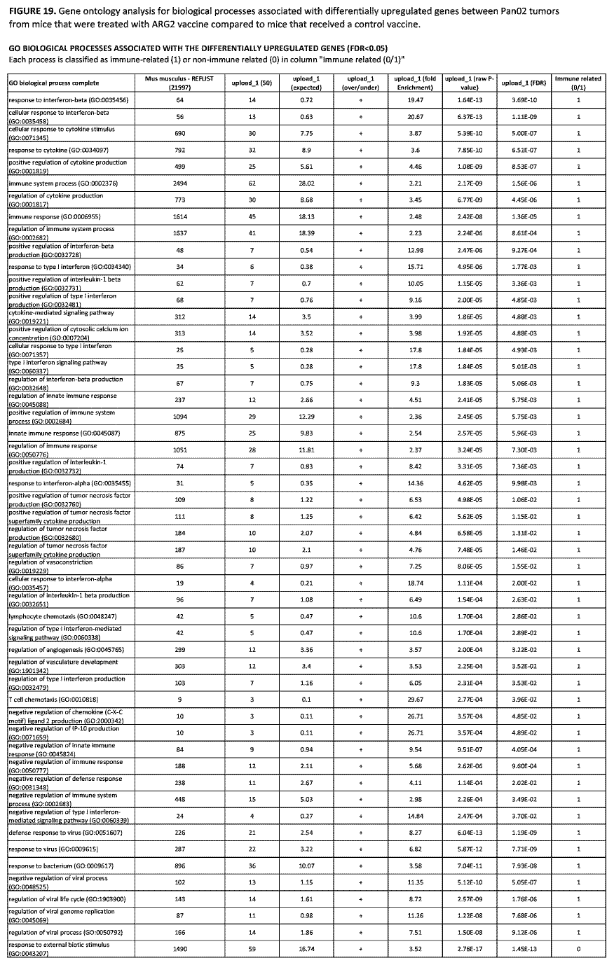

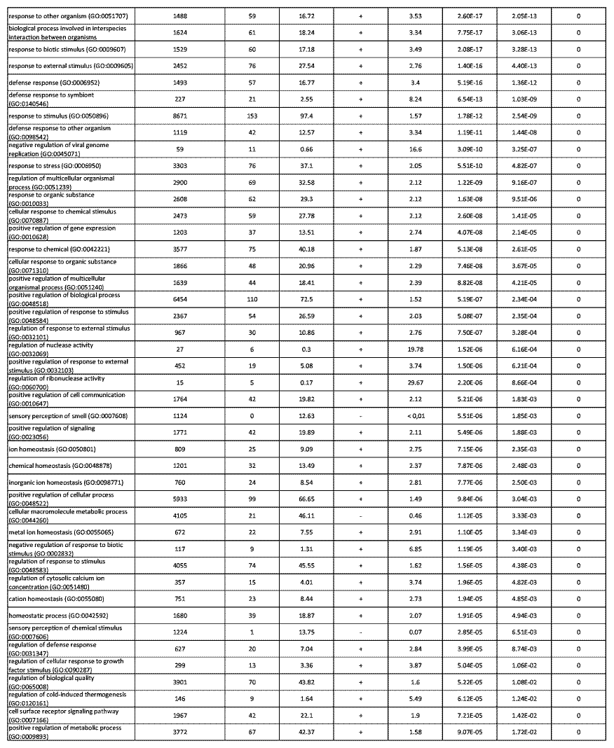

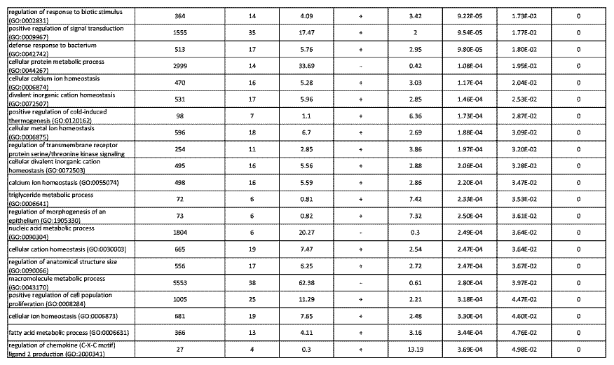

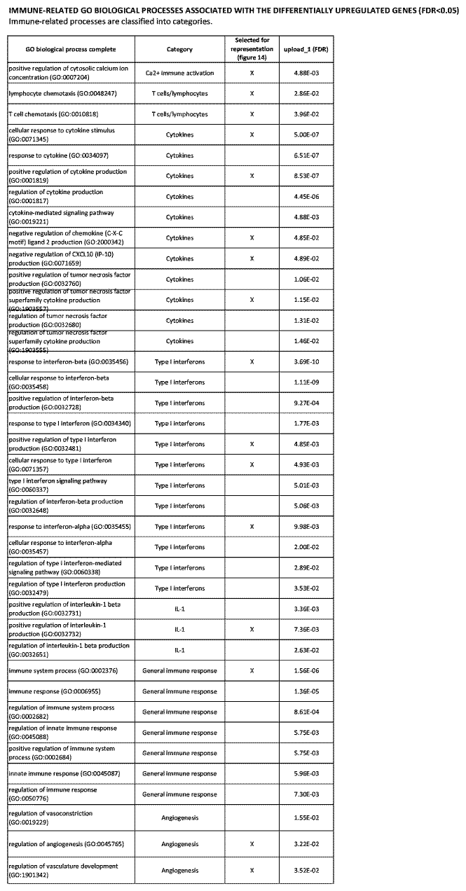

PBMCs were thawed and stimulated with CD3/CD28 Dynabeads (Gibco), following the manufacturer's instructions, in X-vivo supplemented with 10% FBS, 1% sodium pyruvate (Gibco), and 1% non-essential amino acids (Gibco). On days 2, 5, and 7 after bead activation, 300 U/mL IL-2 was added. On day 8, the beads were removed, and the cells were plated in medium without IL-2. On day 9, the cells were stained with CD3-APC/H7, CD4-PerCP, CD25⁻Pe-Cy/7, and the dead-cell stain FVS-510. An aliquot of cells was used as a fluorescence minus one (FMO) control for CD127, while the remaining cells were stained with CD127-FITC. From the CD127-FITC-stained cells, one aliquot was fixed, permeabilized and stained for FOXP3. From the live population, Tregs were sorted as CD3+CD4+CD25^{high}CD127 and Teffs or Trest were sorted as CD3+CD4+CD2510WCD127+. Sorting was performed using a BD FACSMelody™, and data were visualized using BD FACSChorus software. The gating strategy and specific sorting gates are presented in FIGS. 8, 9 and 17, and the antibodies used are listed in Table 2.

### Total RNA Extraction

Cells were harvested, washed in PBS, and pelleted for RNA extraction. Cell pellets were stored at −80° C. until RNA isolation. Total RNA isolation was performed using the RNEasy Plus Mini Kit (Qiagen), following the manufacturer's instructions, with elution in 30 μL RNAse-free water. The RNA concentration was determined using a NanoDrop2000 Spectrophotometer (Thermo Scientific). RNA was stored at −80° C.

cDNA Synthesis and RT-qPCR

cDNA was synthesized using the High Capacity cDNA Reverse Transcription Kit (Applied Biosystems) with random primers and 400-1000 ng RNA as input. Before RT-qPCR analysis, cDNA was diluted 1:2 to 1:4. RT-qPCR analysis was performed using the TaqMan Gene Expression Assay on a Roche Lightcycler 480 instrument. The assay was performed in triplicate, and the results were analyzed as previously described (Bookout et al., Curr. Protoc. Mol. Biol. 73, 15.8.1-15.8.28 (2006)). For low-expression samples with no amplification during the assay, the Ct value was set to 40. Controls lacking reverse transcriptase were included in the primer validation analysis. The primers used in this study are listed in Table 3 below.

TABLE 3
RT-qPCR PRIMERS

| GENE | PRIMER ID | SOURCE |
|---|---|---|
| ARG1 | Hs00163660_ml | Thermo Scientific |
| ARG2 | Hs00982833_ml | Thermo Scientific |
| CTLA4 | Hs00175480_ml | Thermo Scientific |
| FOXP3 | Hs01085834_ml | Thermo Scientific |
| IL-10 | Hs00961622_ml | Thermo Scientific |
| POL2RA | Hs00172187_ml | Thermo Scientific |
| IKZ2 | Hs00212361_ml | Thermo Scientific |
| IL2RA | Hs00907777_ml | Thermo Scientific |
| TNFRSF18 | Hs00188346_ml | Thermo Scientific |

### Preparation of Cell Lysates for Western Blotting

Set2, UKE-1 and sorted Treg and Trest cells were washed twice with sterile PBS before being pelleted and stored at −80° C. Cell pellets were resuspended in ice-cold RIPA Lysis Buffer (Thermo Scientific) supplemented with Halt™ Protease Inhibitor Cocktail (Thermo Scientific) at a 1:100 dilution. Cell lysates were placed under constant agitation for 15 minutes at 4° C. before being centrifuged at 16800×g, 4° C. for 15 minutes. Supernatants were then transferred to new Eppendorf tubes and used for protein concentration measurements performed with the BCA Protein Assay Kit (Thermo Scientific) according to the manufacturer's protocol.

### Western Blot Analysis of ARG2 Expression in Sorted Treg and Trest

Volumes of cell lysate corresponding to 20 μg total protein were mixed with distilled water, Bolt™ Sample Reducing Agent (1:10 dilution) and Bolt™ LDS Sample Buffer (1:4 dilution, Invitrogen) for a total sample volume of 50 μL. The samples were incubated at 99° C. for 10 minutes to aid denaturation and subsequently separated on Bolt™ 4-12% Bis-Tris Plus gels (Invitrogen) for 20 minutes at 200 V using a PowerPac HV (BioRad) and Bolt™ MES SDS Running Buffer (Invitrogen). To allow for protein size quantification, the BioRad Precision Plus Protein Dual Color ladder was utilized. The gel was transferred to an iBlot 2 PVDF Ministack (Invitrogen) and electroblotted with an iBlot Gel 2 Transfer device (Invitrogen) according to the manufacturer's guidelines. The membranes were cut in two pieces to allow for separate stainings of the ARG2 and vinculin proteins. Both parts of the membrane were blocked for 1 hour in TBST buffer (TBS buffer (Thermo Scientific) supplemented with 0.1% Tween-20 (Sigma Aldrich)) with 5% added skimmed milk powder. Next, one part of the membrane was incubated overnight at 4° C. with the primary ARG2-specific antibody diluted 1:1,000 in blocking buffer while the other half of the membrane was kept in TBST overnight. On the following day, the unstained part of the membrane was incubated for 1 hour with the vinculin-specific antibody diluted 1:100,000 in blocking buffer. Following, the membranes were washed with TBST three times for 5 minutes. The ARG2-stained and vinculin-stained membranes were then incubated with either anti-rabbit or anti-mouse secondary antibodies, respectively, at a 1:2,000 dilution in blocking buffer for 1 hour. After three washes, the membranes were developed for 5 minutes with SuperSignal™ West Femto Maximum Sensitivity Substrate (Thermo Scientific) and visualized on a Gel Doc™ XR System (BioRad) using ImageLab software (V.5.2.1). The utilized antibodies are listed in Table 2 above.

### Cytotoxicity Assay

Conventional 51Cr-realease assays were performed to evaluate the cytotoxicity of ARG2-specific CD8⁺ T cells, as previously described in Andersen et al. J. Immunol. 163, 3812-3818 (1999). Briefly, target cells were labelled with 100 μCi radioactive 51Cr for 1 hour, followed by two washes to remove any excess 51Cr outside the cells. Effector cells and target cells were plated at different effector-to-target (E:T) ratios, and incubated for 4 hours. Next, 100 AL supernatant was recovered, and 51Cr release was determined using a 2470 Automatic y-counter (Perkin Elmer). Maximum 51Cr release was determined in separate well by addition of 100 μL of 10% Triton-X to target cells. Spontaneous target cell lysis was determined in other wells by incubating the target cells with medium alone. Assays were set up using technical duplicates for all E:T ratios; maximum and minimum release wells were set up with six technical replicates.

### In Vitro Treg Suppression Assay

The CD8-positive fraction of the activated PBMCs used for Treg/Trest sorting was isolated on day 8 after stimulation initiation using magnetic bead separation (Miltenyi Biotec). The purity of the sorted cells was assessed by flow cytometry after staining the cells with CD3-APC/H7, CD8-FITC and FVS-510. On the following day, the CD8 T cells were stained with carboxyfluorescein succinimidyl ester (CFSE) dye (Sigma Aldrich) at a concentration of 5 μM. Next, 1.5×10⁵ CFSE-labeled CD8 T cells were co-cultured in a round-bottom 96-well plate together with 0.3×10⁵ sorted-purified Treg or Trest cells. The cells were stimulated with Human T activator anti-CD3/CD28 Dynabeads (Gibco) at a ratio of 1:25 and supplemented with 300 U/mL IL-2 one day after co-culture initiation. The proliferation status of the CD8 T cells was analyzed using flow cytometry on day 0 and 5 after initiating the co-culture. Staining the cells with CD4-PerCP enabled segregation of the Treg/Trest cells from the CFSE-labeled CD8 T cells. For each analyzed time-point, three technical replicates were set up. Data were acquired using a BD Canto II flow cytometer and analyzed using FCS Express V.7 and FlowJo V.10. The antibodies used are listed in Table 2 above.

Treg/Trest Population Analysis in Co-Cultures of Activated PBMCs with Effector T Cells

On day 9 following stimulation initiation, 7.5×10⁵ activated PBMCs were incubated alone or together with either 5×10⁵ ARG2-specific CD8 T cells or 5×10⁵ autologous control CD8 T cells. The control CD8 T cells were isolated from activated PBMCs as described above. The cells were cultured for 6 hours in a 48-well plate after which they were stained using the same procedure as for the sorting of Tregs and Trest as described above. In addition, similar gating strategies were utilized (FIG. 9) based on samples that only contained activated PBMCs. The Treg and Trest population sizes of the samples containing ARG2-specific CD8 T cells or control CD8 T cells were compared to the samples containing only activated PBMCs to determine the percentual decrease in the two populations. Three technical replicates were included for both conditions with added T cells while six technical replicates were included for the condition with activated PBMCs alone.

### Animal Experiments

Animal experiments were conducted at the animal facility at Copenhagen University Hospital, Herlev, Department of Oncology. Experiments were performed with approval from the Danish Ethics Committee on Experimental Animal Welfare (Dyreforsøgstilsynet). C57BL/6 female mice were bred in-house from a background of C57BL/6JBomTac. Daily care was performed by animal caretakers in the animal facility.

### Peptide Vaccinations

The murine ARG1 peptide (mARG1_169-177, ISAKDIVYI (SEQ ID NO: 22)) was synthesized by Schäfer, and was dissolved in DMSO to a stock concentration of 10 mM. The murine ARG2 peptide (mARG2_188-196, LSPPNIVYI (SEQ ID NO: 21)) was synthesized by PepScan or Schäfer and dissolved in sterile water to a stock concentration of 2 mM. The ARG2 peptide was filtered before use by passing it through a sterile 0.22 μm filter. The purity of all synthesized peptides was >90%. All peptides used in this study are listed in Table 1. ARG2 peptide (100 μg) was suspended in a volume of 50 μL sterile water and emulsified 1:1 with Montanide ISA 51 (Seppic). Control vaccines included 50 μL sterile water, emulsified 1:1 with Montanide. Mice were vaccinated subcutaneously at the base of the tail with 100 μL of control- or peptide emulsions. For tumor studies, mice were vaccinated on day 11 and 17 (Pan02), on day 0 and 7 (MC38) or on day 0 and 5 (B16-F10 and LL2) post tumor inoculation. For the remaining studies, mice were vaccinated 1-3 times with a minimum 1-week interval between vaccinations.

### Tumor Studies

Pan02, MC38, B16-F10 and LL2 cell lines were thawed one week prior to inoculation and cultured in DMEM (Gibco) with 10% fetal bovine serum (FBS) (Invitrogen) and 1% penicillin and streptavidin (P/S) (Life Technologies).

Female C57BL/6 mice received subcutaneous inoculations of 5×10⁵ Pan02, MC38, B16-F10 or LL2 cells on the right flank. The tumor volume was measured with a digital caliper and calculated as (length×width²)/2. Experimental endpoint was defined as tumor volume above 1000 mm3 or the presence of tumoral ulceration. Investigators that performed tumor measurements were blinded to the treatment groups.

### Murine IFNγ ELISPOT

Mice were sacrificed at endpoint for tumor studies and one week after the last vaccination for the remaining studies. Spleens were harvested, smashed through a 70 μm filter, and red blood cells were lysed with Red Blood Cell Lysis Solution (Qiagen). 8×10⁵ splenocytes were plated per well in IFNγ ELISPOT plates. IFNγ ELISPOT was performed as previously described. Tumors were harvested, cut into smaller pieces and enzymatically digested in RPMI medium containing 2.1 mg/ml collagenase type 1 (Worthington), 75 μg/ml DNase I (Worthington), 5 mM CaCl2, and 1% P/S. Cells were filtered through a 70 μm cell strainer and red blood cells were lysed as described above. CD45+tumor infiltrating lymphocytes (TILs) were isolated from tumor single-cell suspensions with CD45 microbeads (for MC38 and LL2 tumors) (Miltenyi Biotec) or with CD45 (TIL) microbeads (for B16-F10 tumors) (Miltenyi Biotec). The isolated CD45+ cells were rested overnight. 5×10⁵ CD45+ cells were plated per well in IFNγ ELISPOT plates. For phenotyping ARG2 responses, CD8+ and CD4⁺ T cells were isolated from the spleen of vaccinated mice with CD8a (Ly-2) and CD4 (L3T4) microbeads (Miltenyi Biotec), respectively, following manufacturer's instruction. Isolated T cells were rested overnight. 2.8×10⁵ CD8+ or CD4⁺ T cells were plated in an IFNγ ELISPOT plate with 6×10⁵ splenocytes from a naïve mouse (used as antigen-presenting cells). Peptide-specific responses were reported as the difference in the average number of spots between peptide-stimulated and unstimulated wells.

### Tumor RNA Extraction and RNA Sequencing

Tumor fragments (≤30 mg) were stored in RNAlater (Invitrogen) at −80° C. Tumors were homogenized on a TissueLyser (Qiagen) and RNA was extracted with the RNEasy Plus Mini Kit (Qiagen), according to the manufacturer's instructions. RNA concentration was measured on a Nanodrop 2000 Spectrophotometer. Isolated RNA was stored at −80° C. RNAseq was performed on tumors from four untreated and six vaccinated mice as previously described in Fjæstad et al., Oncogene 41, 1364-1375 (2022). In short, 500 ng purified RNA (RIN score>7) was enriched for polyadenylated mRNA using oligo dT magnetic beads (Illumina) followed by fragmentation and cDNA synthesis using random priming (NEBNext). The cDNA was prepared for Illumina sequencing by adaptor ligation followed by indexing using PCR and size selection. Concentration of the cDNA libraries was determined by the KAPA Library Quantification Kits (Roche) and sequenced using Novaseq 6000 Illumina sequencing platform. RNAseq data is available on GEO repository (GSE212500). Alignment and quantification of reads was performed as previously described in Fjæstad et al. Shortly, sequenced DNA was aligned to the GRCm39 reference genome assembly using STAR-v2.7.8 and reads were quantified using featureCounts and the ENSEMBL genes and transcripts version 104. Differential gene expression was analysed by DESeq2. Volcano plots were generated with EnhancedVolcano package (v1.8.0) based on the differential gene expression analysis. Biological processes associated with differentially upregulated genes was assessed with The Gene Ontology Resource (http://geneontology.org/) followed by classification of the most specific sub-processes.

### ImmuCC

The computational framework of the CIBERSORT analytical tool, along with the developed ImmuCC signature matrix (non-tissue specific), suitable for the deconvolution of mouse bulk RNA-Seq data, were used to characterize and to quantify 25 immune cell subtypes. The ImmuCC signature matrix used consists of 511 genes of which 510 genes from our bulk RNA-Seq data was mapped (1 missing). For the deconvolution of the bulk RNA-Seq samples with CIBERSORT, DEseq2's median of ratios normalized data were used to produce the input mixture matrix. Additionally, the analysis included both CIBERSORT-Relative and CIBERSORT-Absolute modes. While CIBERSORT-Relative represents immune cell fractions relative to the total immune content, thus suitable for intra-sample comparisons, CIBERSORT-Absolute produces a score that quantifies the abundance of each cell type, making it appropriate for intra-sample comparisons between cell types as well as inter-sample comparisons of the same cell type. The CIBERSORT outputs were generated by performing 1000 permutations and by disabling the quantile normalization parameter. For this study, two population schemes were defined (compact and extended, Table 5 and Table 6), resulting in the aggregation of some of the 25 immune sub-populations. Total absolute scores for sub-populations merged were calculated as the sum of the sub-populations. The relative fractions were re-calculated based on each scheme's new total immune content.

### Statistical Analysis

Statistical analysis of ELISPOT responses was performed in R studio using distribution free resampling rule (DFR) as described by Moodie et al. (Methods Mol. Biol. 792, 185-196 (2012)). The descriptive statistics from the proliferation assay and the change in Treg population size from the Treg/Trest population analysis was analyzed statistically using unpaired t-tests. 2-way ANOVA with comparisons between multiple time-points was performed to determine statistical significance for tumor growth curves. Statistical significance for the difference in peptide-specific IFNγ-secreting cells and ImmuCC-related analysis between treatment groups were assessed with a Mann-Whitney U test and an unpaired, two-tailed t test, respectively. Statistical analyses were performed in GraphPad Prism (version 9).

### Example 2—Spontaneous CD8⁺ T-Cell Responses to ARG2

The inventors investigated ARG2-specific CD8⁺ T cells using short peptides that could be directly presented on MHC class I molecules. In silico HLA-prediction algorithms (available at syfpeithi.de and cbs.dtu.dk) were used to generate a library of 15 short ARG2-derived peptides (9mers and 10mers) predicted to strongly bind to HLA-A2, as indicated in Table 4 below.

TABLE 4
Peptide library HLA-A2 predicted
AR2-derived peptides

| PEP- | SEQ | | | PRE- | SCORE (SYFP) |
|---|---|---|---|---|---|
| TIDE | ID | POSITION | | DICTION | OR RANK |
| NAME | NO: | (AA) | SEQUENCE | ALGORITHM | (NETMHC) |
| A2S01 | 1 | 2-10 | SLRGSLSRL | SYPF & | 28 (SYFP) |
| | | | | NetMHC | and 1.10 |
| | | | | | (NetMHC) |
| A2S02 | 2 | 10-18 | LLQTRVHSI | SYFP | 27 |
| A2S03 | 3 | 18-26 | ILKKSVHSV | SYPF & | 28 and 1.50 |
| | | | | NetMHC | |
| A2S04 | 4 | 53-61 | GLMKRLSSL | SYPF & | 28 and 0.250 |
| | | | | NetMHC | |
| A2S05 | 5 | 84-92 | NLIVNPRSV | SYFP | 24 |
| A2S06 | 6 | 94-102 | LANQELAEV | SYFP | 25 |
| A2S07 | 7 | 114-122 | VTLGGDHSL | SYFP | 24 |
| A2S08 | 8 | 166-175 | FLLRELQDKV | NetMHC | 0.400 |
| A2S09 | 9 | 167-175 | LLRELQDKV | SYFP | 27 |
| A2S10 | 10 | 170-178 | ELQDKVPQL | SYFP | 24 |
| A2S11 | 11 | 193-201 | IVYIGLRDV | SYFP | 23 |
| A2S12 | 12 | 229-237 | KVMERTFDL | NetMHC | 0.400 |
| A2S13 | 13 | 278-286 | YIAEEIHNT | SYPF & | 23 and 1.10 |
| | | | | NetMHC | |
| A2S14 | 14 | 287-295 | GLLSALDLV | SYPF & | 27 and 0.125 |
| | | | | NetMHC | |
| A2S15 | 15 | 288-297 | LLSALDLVEV | SYPF & | 0.300 |
| | | | | NetMHC | |

These 15 peptides (A2S01 to A2S15) were screened in peripheral blood mononuclear cells (PBMCs) from five healthy donors (HD), who were confirmed to be HLA-A2 positive (HLA-A2⁺). PBMCs were stimulated with each peptide in vitro and restimulated in IFNγ ELISPOT after 12-14 days of culture.

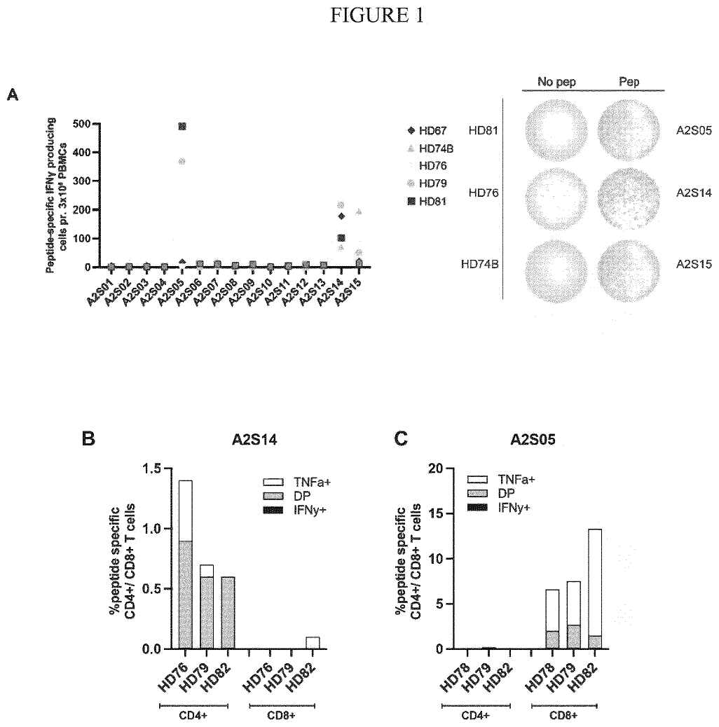

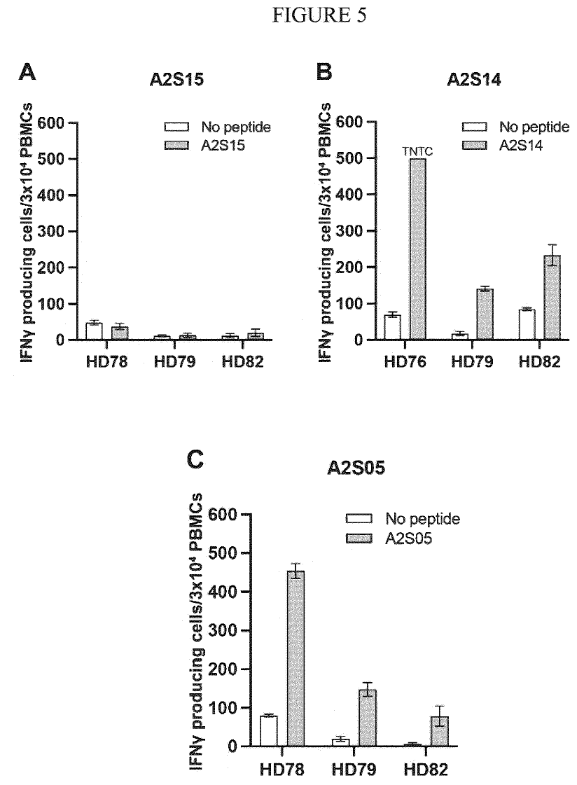

Peptides A2S05 (SEQ ID NO: 5), A2S14 (SEQ ID NO: 14) and A2S15 (SEQ ID NO: 15) were identified as strong candidates, with each eliciting significant responses in PBMCs from three or more donors (FIG. 1A). However, the responses seen with A2S15 could not be validated, as repetition of the IFNγ ELISPOT in HDs did not show significant responses (FIG. 5A). On the other hand, A2S14 elicited strong IFNγ ELISPOT responses in the validation setup (FIG. 5B). Further characterization of the responses by intracellular cytokine staining (ICS) for TNFα and IFNγ revealed that the reaction was from CD4⁺ T cells (FIG. 1B). A2S05 was also validated to elicit strong IFNγ ELISPOT responses (FIG. 5C) and ICS demonstrated that the responses were from CD8⁺ T-cells (FIG. 1C). A2S05 was thus identified as an ARG2 peptide that elicited CD8⁺ T-cell responses.

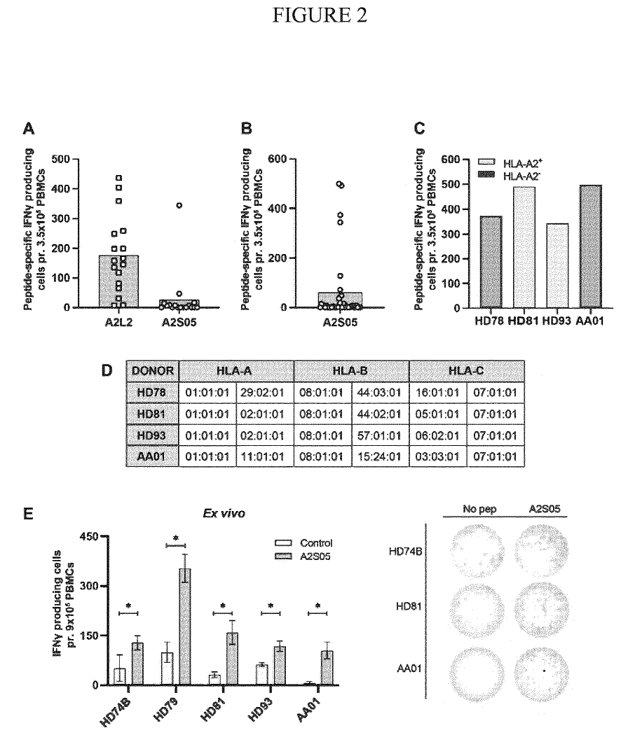

Next, 17 HDs were screened for response to A2S05 and to the previously described highly immunogenic ARG2-derived peptide A2L2 (SEQ ID NO: 17). Strong and frequent responses to A2L2 were observed. However, only few responses to A2S05 were observed in this particular assay (FIG. 2A). The HDs used for this assay were of undetermined HLA-type. It was therefore proposed that the screened donors could have HLA-types that were incompatible with peptide A2S05. Accordingly, a further 38 donors (13 cancer patients with solid tumors and 25 healthy donors) with known HLA-A2 status (positive or negative) were screened for response to A2S05. Most donors were selected on the basis of being HLA-A2+ (n=29) but some HLA-A2″ donors (n=9) were included as controls. Again, responses to A2S05 were not frequently observed, with statistically significant responses occurring in only 10 out of the 38 donors (FIG. 2B). Interestingly, responses to A2S05 were observed in both HLA-A2+ and HLA-A2-donors (FIG. 2C), demonstrating that A2S05 was not HLA-A2 restricted as suggested by the in silico HLA-prediction algorithm.

Full HLA-typing of four donors with strong A2S05 responses was the performed. It was found that all four donors shared HLA-A1, HLA-B8 and HLA-C7 (FIG. 2D). Importantly, in some donors with strong in vitro responses to A2S05, strong and significant responses in PBMCs ex vivo were also detected (FIG. 2E).

### Example 3—Characterization of ARG2-Specific CD8⁺ T Cells

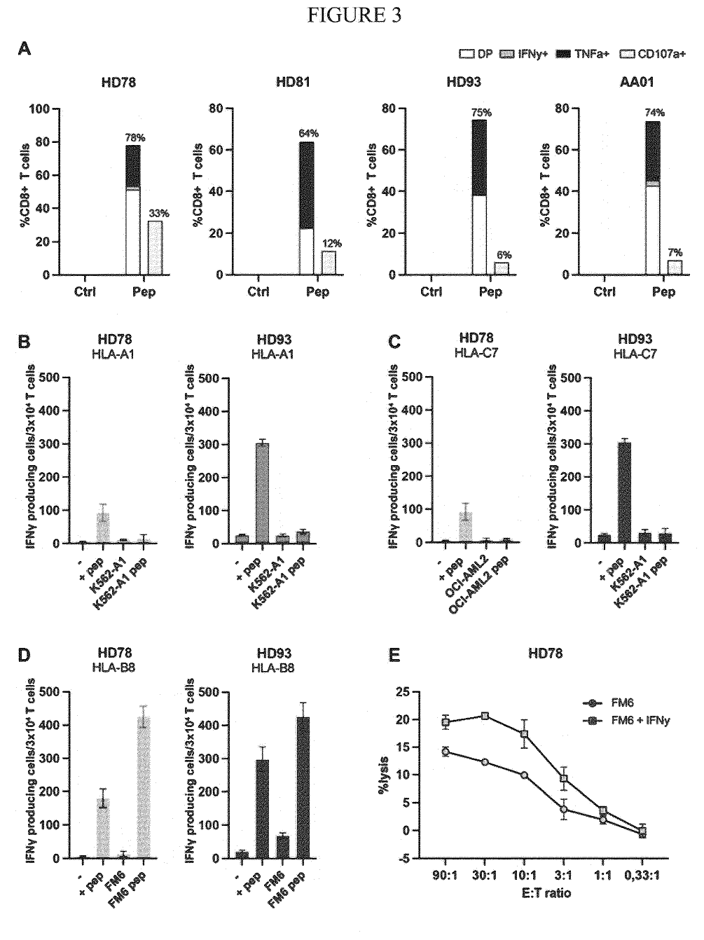

To further characterize the ARG2-specific CD8⁺ T cells, A2S05-specific T-cell cultures from four donors were established. Briefly, donor PBMCs were stimulated with A2S05 in vitro. 12 days later, peptide-specific T cells were re-stimulated and isolated using an IFNγ capture kit. IFNγ-producing cells were expanded using a rapid expansion protocol and the specificity was examined at 16-17 days after expansion by ICS for IFNγ and TNF. Highly specific CD8+cultures from all four donors were established (FIG. 3A).

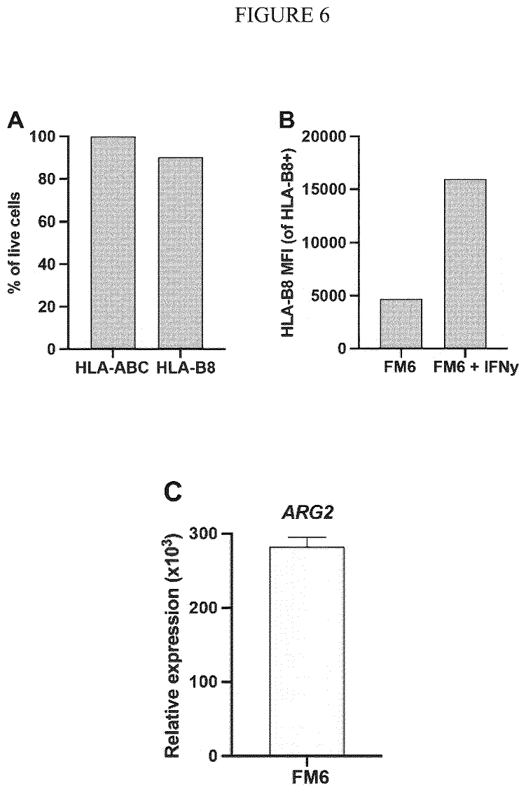

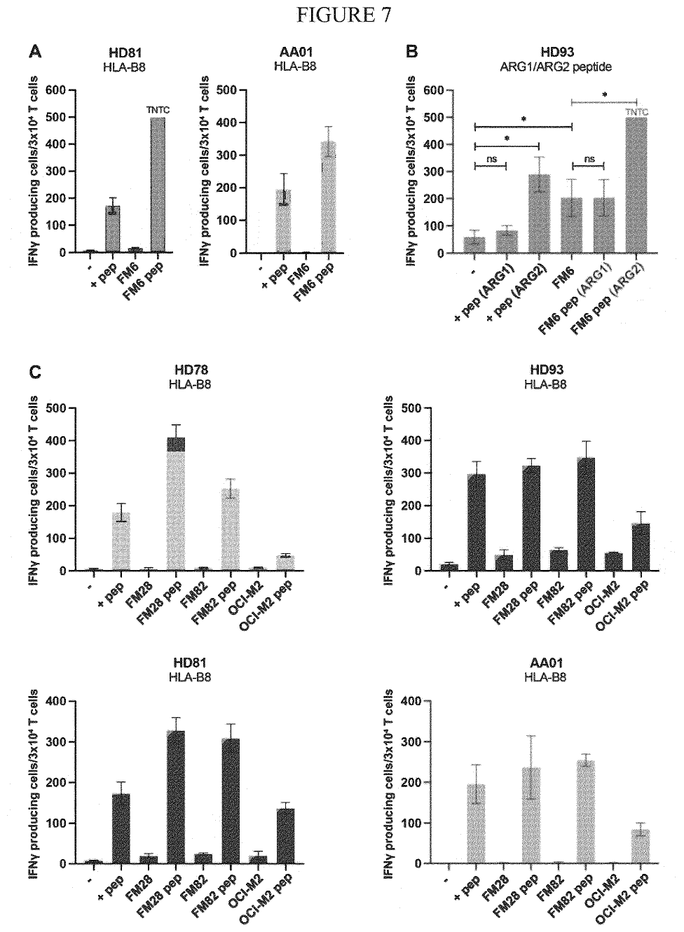

The established cultures were used to determine the HLA-restriction of A2S05. IFNγ ELISPOTs were performed using A2S05-specific T cells from HD78 and HD93 co-cultured with different target cells lines pulsed with A2S05 peptide. These results revealed no reactivity toward HLA-A1⁺ and HLA-C7⁺ cell lines (FIGS. 3B and 3C), but recognition and reactivity activity toward the HLA-B8⁺ metastatic malignant melanoma cell line FM6 (FIG. 3D). FM6 was confirmed to be HLA-B8⁺ by flow cytometry and confirmed to express ARG2 by RT-qPCR (FIG. 6). The two remaining ARG2-specific CD8⁺ T-cell cultures were confirmed to show reactivity against FM6 (FIG. 7A) and all four ARG2-specific CD8⁺ T-cell cultures were confirmed to show reactivity against three additional HLA-B8⁺ cell lines (FIG. 7C). The specificity of the ARG2-specific CD8 T-cell cultures was confirmed by the lack of reactivity against a peptide with the corresponding ARG1 sequence (ARG1_65-73) in IFNγ ELISPOT (FIG. 7B).

Next, standard 51Cr cytotoxicity assays were performed to establish whether the ARG2 specific CD8⁺ T cells could lyse FM6 cells. Indeed, the specific T cells lysed FM6 cells in a concentration-dependent manner (FIG. 3E), thus demonstrating the cytotoxic capacity of ARG2-specific CD8⁺ T cells. Stimulation with IFNγ increased the mean fluorescence intensity (MFI) of HLA-B8 expression (FIG. 6) and resulted in a small increase in the lysis of FM6 cells (FIG. 3E).

Thus, the HLA-B8 restriction of A2S05 peptide was confirmed by specific recognition and cytolytic activity against the metastatic malignant melanoma cell line FM6 and three other HLA-B⁺ cancer cell lines. This was surprising given that the peptide had been predicted to bind HLA-A2. Interestingly, it has been proposed that the HLA-B8 haplotype plays a protective role against melanoma, based on the observation of significantly decreased frequency of HLA-B8 in advanced melanoma patients compared to in healthy donors (Fensterle et al., BMC Med. 4, 1-6 (2006)). Moreover, in chronic myeloid leukemia (CML) HLA-B8 expression is associated with decreased incidence of CML (Posthuma et al., Blood 93, 3863-3865 (1999)). ARG2 upregulation has also been observed in Tregs and malignant melanoma cells in metastatic melanoma (Lowe et al., JCI Insight 4, (2019) and Yu et al., J. Cell. Physiol. 235, 9997-10011 (2020)) and an immunosuppressive role has been described for ARG2 in acute myeloid leukemia (Mussai et al., Blood 122, 749-758 (2013). Without being bound by theory, the HLA-B8 restriction of a class I ARG2 peptide implies that ARG2-specific T cells play a role in immune surveillance in melanoma and CML.

### Example 4—ARG2-Specific T Cells Specifically Recognize Activated Regulatory T Cells

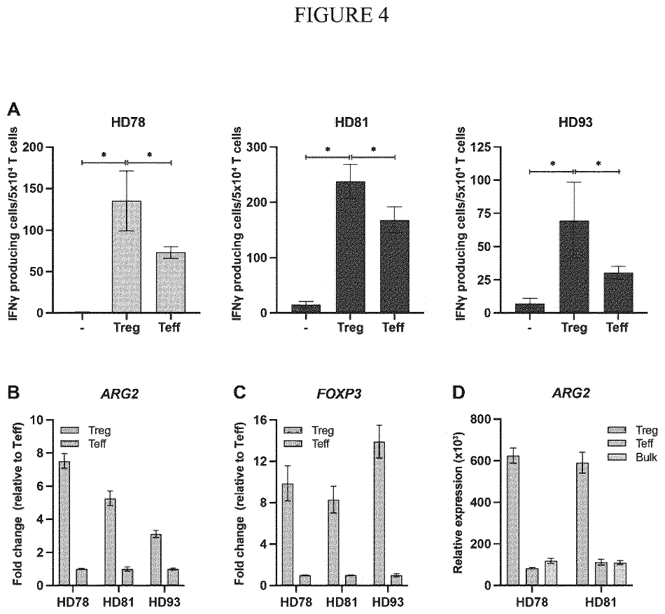

In a recent study, Lowe and colleagues found that activated Tregs from peripheral blood exhibited high ARG2 expression when compared to activated effector CD4⁺ T cells (Teffs) and demonstrated an ARG2-dependent suppression of T-cell proliferation by Tregs (Lowe et al., JCI Insight 4, (2019)). It was hypothesized that if activated Tregs express higher ARG2 levels than activated Teffs, they could be preferentially recognized by ARG2-specific T cells. To test this hypothesis, PBMCs were activated using CD3/CD28 beads, and added IL-2 on days 2, 5 and 7 based on the experiments described by Lowe et al. On day 9 after activation, purified Tregs and Teffs were purified using FACS (FIGS. 8A and 9). The purified Tregs or Teffs were then plated as target cells for autologous ARG2-specific T cells in an IFNγ ELISPOT assay. Significantly higher responses to Tregs than to Teffs were observed in all three donors (FIG. 4A) and RT-qPCR analysis showed 3-7 fold higher ARG2 expression in Tregs than in Teffs (FIG. 4B) along with minimal levels of ARG1 expression (FIG. 8E), thus indicating ARG2-dependent recognition. The Treg phenotype was confirmed by preferential expression of Treg signature genes, including FOXP3 (FIG. 4C), IL-10 and CTLA-4 (FIGS. 8C-D) in Tregs. Finally, ARG2 expression levels in Tregs and Teffs were compared to expression levels in the bulk culture of activated cells from which the two subsets were isolated and found the bulk culture to express ARG2 at levels comparable to Teffs (FIG. 4D).

The ability of ARG2-specific T cells to recognize and react to Tregs highlights their immune modulatory function has thus been demonstrated. The ARG2-specific T cells ability to target regulatory cells indicates that ARG2-specific T cells are anti-Tregs, characterized by their ability to “regulate the regulators”. The preferential expression of ARG2 in activated Tregs compared to the activated bulk culture or activated Teffs suggests that ARG2 induction serves as an active mechanism for increased immunosuppressive capacities of Tregs. Therefore, without being bound by theory, the targeting of activated Tregs by ARG2-specific T cells could reasonably be predicted to have important immune modulatory potential by removing the immune suppression exerted by activated Tregs with high ARG2 expression. Many tumors are characterized by high numbers of such Tregs and the targeting of activated immunosuppressive Tregs by ARG2-specific anti-Tregs may therefore relieve Treg-mediated immune suppression of tumor-infiltrating T cells (TILs).

### Example 5—ARG2-Specific T Cells Specifically Recognize Activated Regulatory T Cells

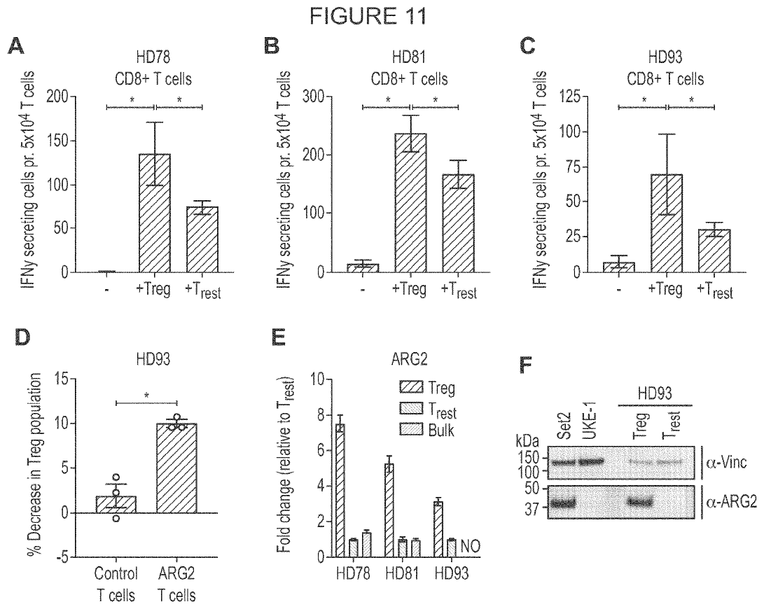

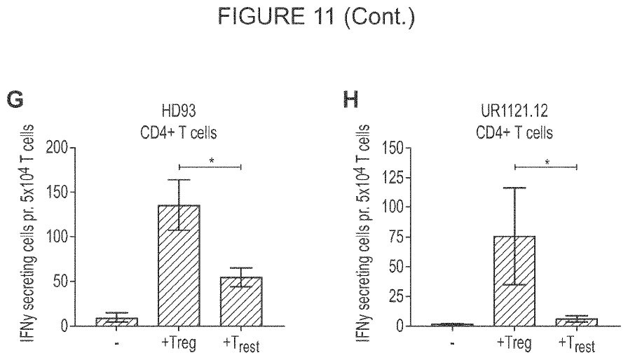

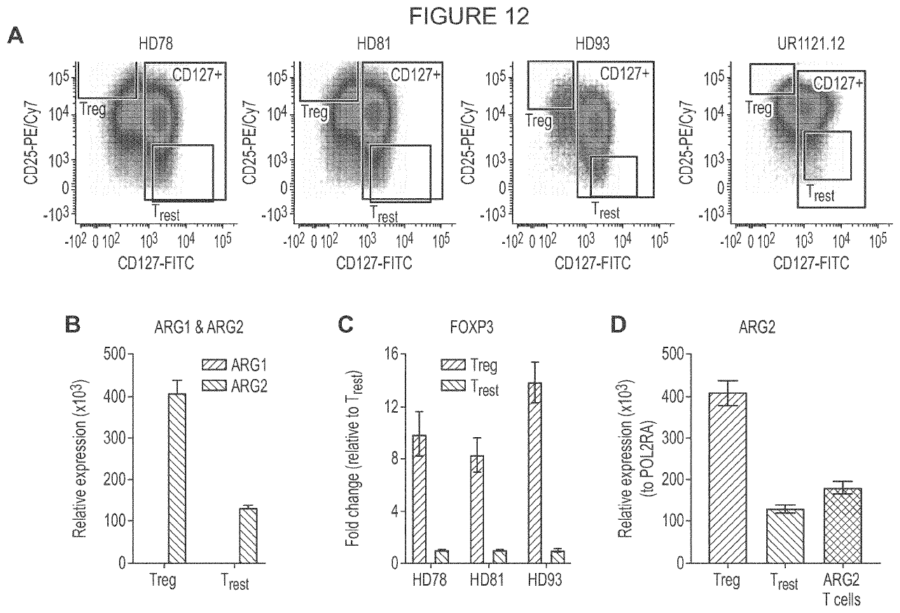

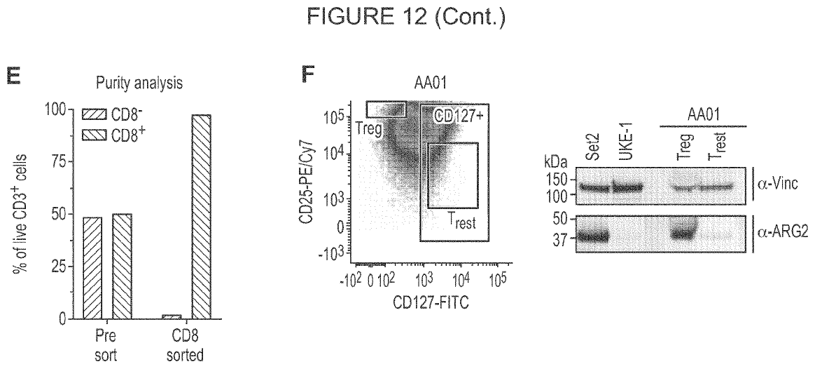

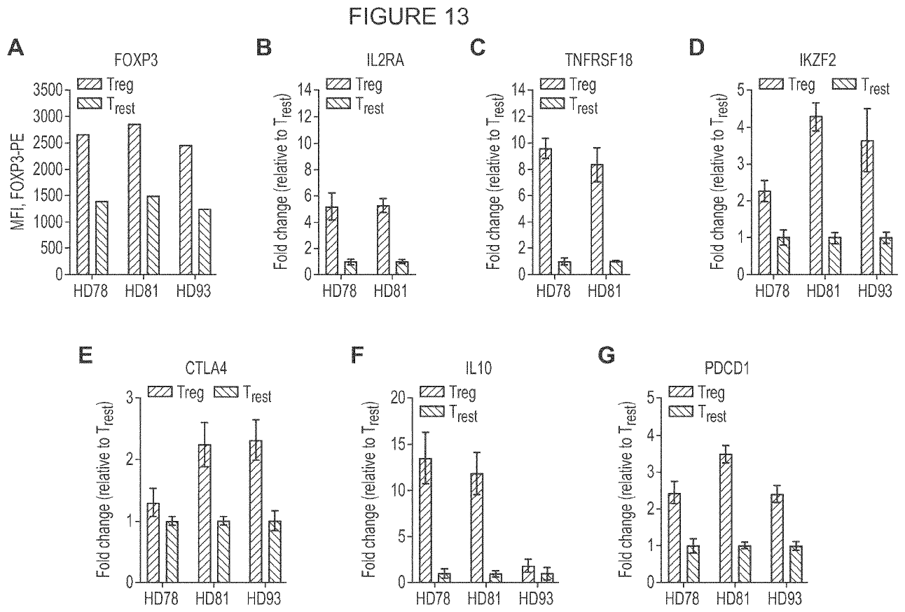

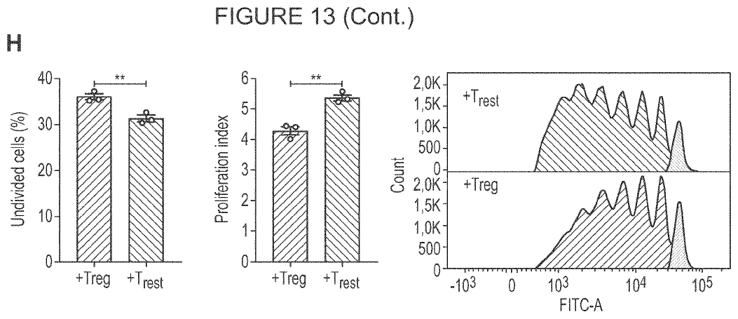

In a recent study, Lowe and colleagues found that activated Tregs from peripheral blood exhibited high ARG2 expression when compared to activated effector CD4⁺ T cells (Teffs) and demonstrated an ARG2-dependent suppression of T-cell proliferation by Tregs (Lowe et al., JCI Insight 4, (2019)). It was hypothesized that if activated Tregs express higher ARG2 levels than other T cells, they could be preferentially recognized by ARG2-specific T cells. To test this hypothesis, PBMCs were activated using CD3/CD28 beads, and IL-2 was added on days 2, 5 and 7 based on the experiments described by Lowe et al. On day 9 after activation, purified Tregs and resting T cells (Trest) were purified using FACS (FIGS. 12A and 13). The purified Tregs or Trest were then plated as target cells for autologous ARG2-specific T cells in an IFNγ ELISPOT assay. Significantly higher responses to Tregs than to Trest were observed in all three donors (FIG. 11A-C). Furthermore, co-culture of activated PBMCs with ARG2-specific T cells led to a significant decrease in the Treg population when compared to activated PBMCs co-cultured with autologous CD8⁺ T cells (FIG. 11D) suggesting cytotoxic activity of ARG2-specific T cells towards Tregs. Importantly, RT-qPCR analysis showed 3-7 fold higher ARG2 expression in Tregs than in Trest (FIG. 11E) along with minimal levels of ARG1 expression (FIG. 12B), indicating ARG2-dependent recognition of Tregs. Preferable ARG2 expression in Tregs on protein level was confirmed by western blot analysis (FIGS. 11F and 12F). ARG2 expression levels in Tregs and Trest were also compared to expression levels in bulk culture of activated PBMCs from which the two T-cell subsets were isolated and found the bulk culture to express ARG2 at levels comparable to Trest (FIG. 11E). Moreover, ARG2 expression in the ARG2-specific T cells was comparable to the ARG2 expression in Trest (FIG. 12D). Finally, Tregs or Trest were plated as target cells for autologous ARG2-specific CD4⁺ T cells in an IFNγ ELISPOT assay. Interestingly, ARG2-specific CD4⁺ T cells also showed significantly higher responses toward Tregs than Trest (FIG. 11F-G).

To confirm the immunosuppressive phenotype of the isolated, ARG2-expressing Tregs, an aliquot of activated PBMCs was stained intracellularly for FOXP3. Assessment of FOXP3 expression between cells sorted as Tregs and Trest showed higher FOXP3 expression in Tregs compared to Trest (FIG. 13A). This was also confirmed by RT-qPCR analysis of FOXP3 expression in Tregs and Trest (FIG. 12C). To further validate the sort purification strategy, the expression of several other Treg signature genes was assessed by RT-qPCT and it was found that IL2RA (CD25), TNFRSF18 (GITR), IKZF2 (HELIOS), CTLA4, and IL10 are preferentially expressed in Tregs (FIG. 13B-F). Interestingly, Tregs also preferably expressed PDCD1 (PD-1) over Trest (FIG. 13G). Finally, the functional phenotype of the sort-purified Tregs was confirmed by a proliferation assay. To this end, highly pure populations of autologous CD8⁺ T cells were isolated (FIG. 12E). Proliferation of anti-CD3/CD28 and IL-2 stimulated CD8⁺ T cells co-cultured with Trest or Tregs was assessed over the course of 5 days and it was found that Tregs significantly suppressed CD8⁺ T cell proliferation compared to Trest (FIG. 13H).

### Example 6-ARG2-Specific T Cells Induced by Peptide Vaccination are Immune Modulatory and Inhibit Tumor Growth in a Murine Model of Pancreatic Cancer

The present inventors previously identified a highly immunogenic murine ARG2-derived epitope (mARG2_188-196, LSPPNIVYI) that elicited strong and frequent T-cell responses upon a single vaccination. It was shown that vaccination with the immunogenic ARG2-derived peptide induced tumor growth delay in a murine model of lung cancer. To confirm the anti-tumor effect of ARG2-derived peptide vaccination in another tumor model and to investigate the immune modulatory function of ARG2-specific T cells, the syngeneic mouse model Pan02 was used. Pan02 is a model of pancreatic ductal adenocarcinoma (PDAC), and thereby relevant for studying ARG2-based vaccines given the previously demonstrated correlation between ARG2 expression and poor prognosis in patients with PDAC together with the demonstration of high Arg2 expression in Pan02 tumors at tumor endpoint.

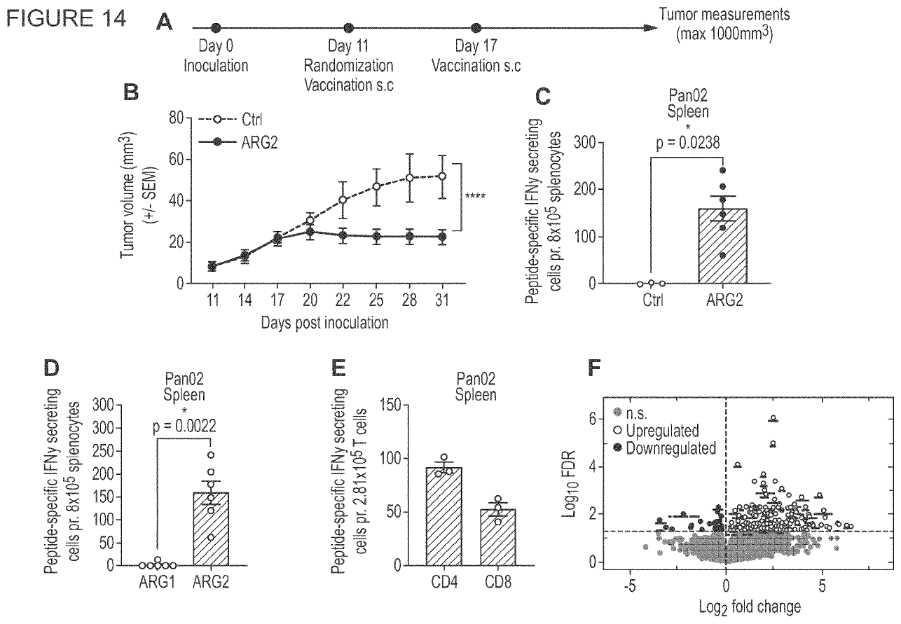

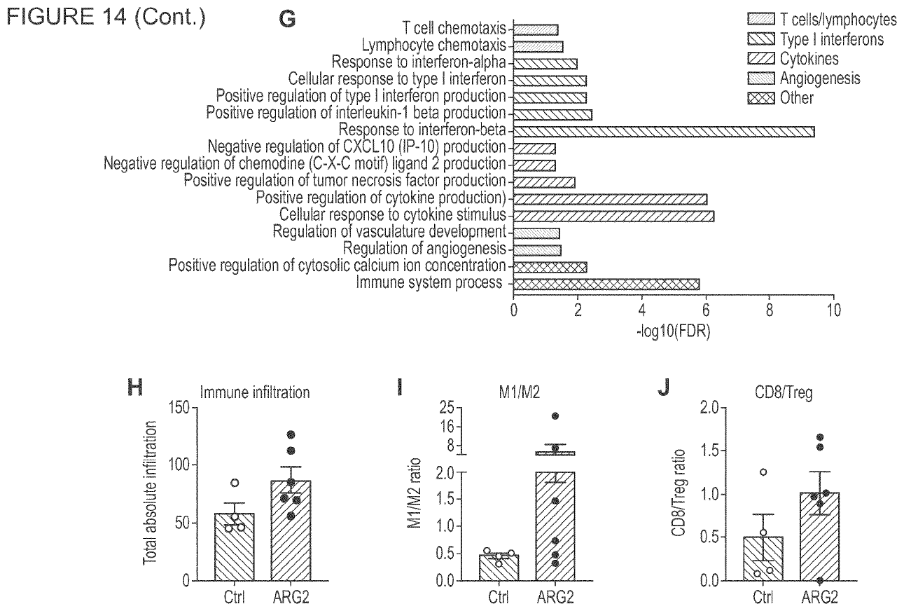

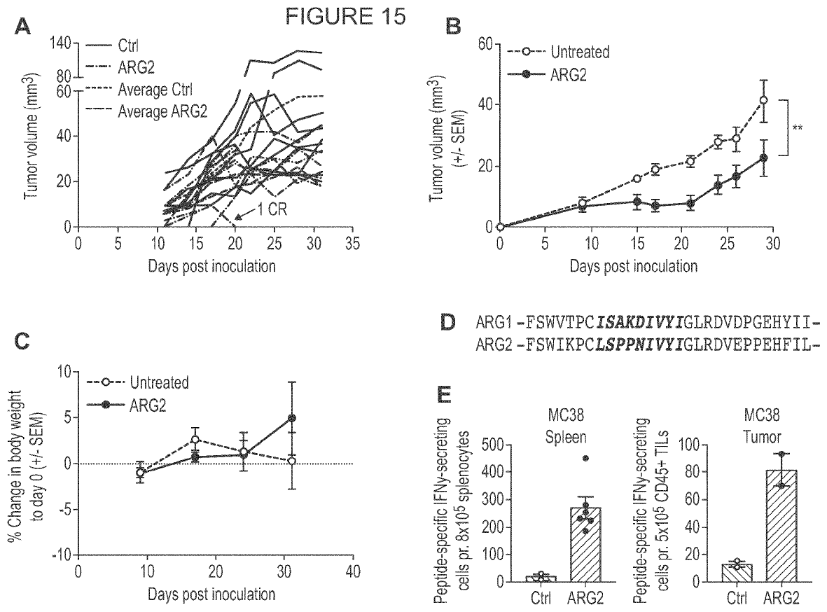

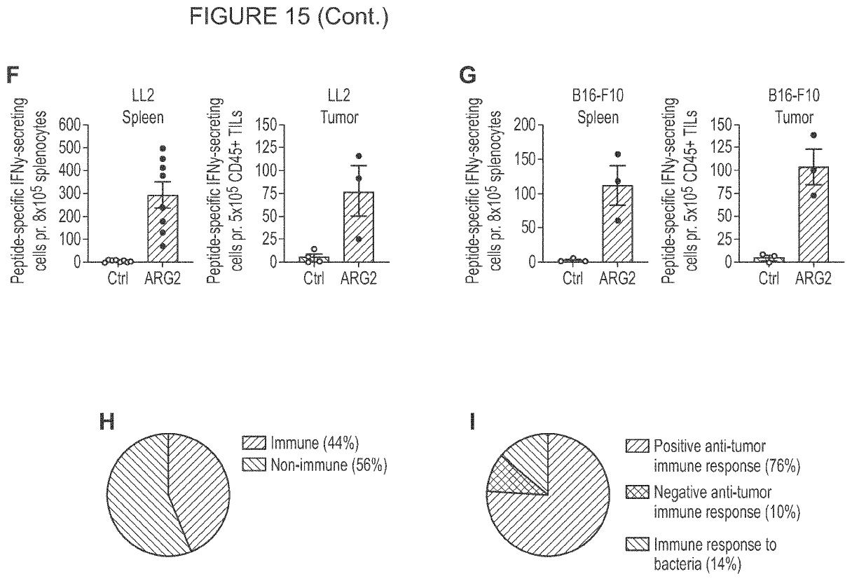

C57BL/6 mice were challenged with Pan02 cells and on day 11, when tumors became palpable, mice with similar average tumor volumes were allocated into different treatment groups. The mice received either the previously described ARG2-based vaccine, or a control vaccine on days 11 and 17 post tumor inoculation (FIG. 14A). Intriguingly, marked inhibition of tumor growth was observed in the ARG2-vaccinated mice; indeed, tumor growth ceased after day 20 (FIG. 14B). Moreover, one mouse in the ARG2-vaccinated group showed complete tumor regression (FIG. 15A). These results were confirmed in an independent study (FIG. 15B). The safety of the vaccine was also demonstrated with no decrease in bodyweight in the ARG2-vaccinated group (FIG. 15C).

Six ARG2-vaccinated mice and three control-vaccinated mice were randomly selected for ELISPOT analysis on day 31 post tumor inoculation. Here, strong responses in all ARG2-vaccinated mice were observed, with no responses in control-vaccinated mice (FIG. 14C). The observed immune responses were specific to ARG2 as no cross-reactivity to a peptide derived from the corresponding region of ARG1 (mARG1_169-177, ISAKDIVYI) was observed, despite partly similar sequences (FIGS. 14D and 15D). Next, pairs of ARG2-vaccinated mice were randomly selected and pooled splenocyte samples were prepared. From these samples, CD4+ and CD8⁺ T cells were isolated. In ELISPOT assays, responses were observed in both the CD4+ and the CD8+ fractions in all three groups of ARG2-vaccinated mice (FIG. 14E).

Due to the small tumor sizes, limited amounts of tumor tissue were available. However, homing of ARG2-specific T cells to the tumor of mice vaccinated with the ARG2-based vaccine was shown in three other murine tumor models (FIG. 15E-G), suggesting that the vaccine induces tumor infiltration of ARG2-specific T cells upon vaccination. To assess the consequences of inducing ARG2-specific T cells on the transcriptional landscape in the tumor, whole tumor RNA was isolated from 4 mice treated with the control vaccine and 6 mice treated with the ARG2-based vaccine and RNAseq was performed. Differential gene expression analysis identified a total of 282 upregulated and 33 downregulated genes after vaccination with ARG2-peptide (FIG. 14F). Gene ontology analysis based on biological processes showed that within the genes upregulated in mice treated with the ARG2-based vaccine, 44% of the enriched processes were associated with tumor immunology (FIG. 15H). Of these, 76% were associated with positive anti-tumor immune responses, 10% were associated with negative anti-tumor immune response and the remaining 14% were associated with immune responses to bacteria (FIG. 15I). Among the significantly enriched biological processes in mice treated with the ARG2-based vaccine, processes associated with both innate- and adaptive immunity were found. An enrichment of processes associated with T cells and lymphocytes, type I interferons, cytokine regulation, angiogenesis and immune system processes and calcium ion concentrations were observed, altogether indicating the development of an immune responses in the ARG2-vaccinated mice (FIG. 14G).

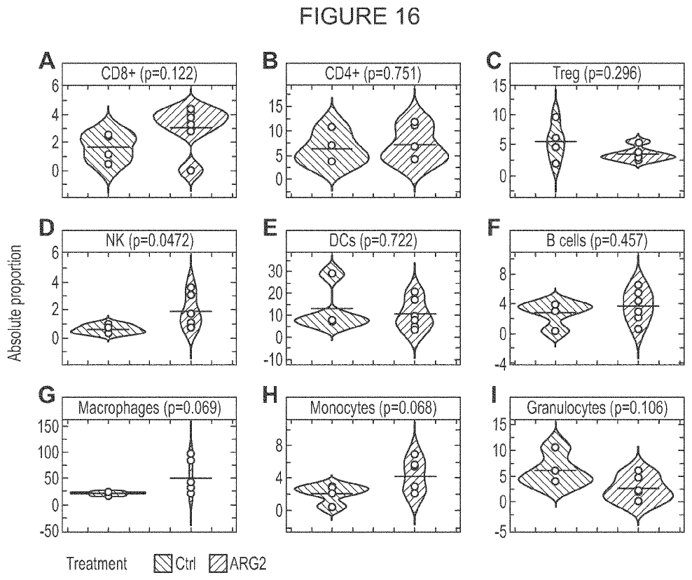

Following these observations, the ImmuCC algorithm was used to assess the relative composition of infiltrating immune cell types in the bulk tumor samples (Table 5 and 6). Overall, there was a clear trend towards a higher average infiltration of immune cells into the tumor microenvironment upon vaccination with the ARG2-based immune modulatory vaccine (FIG. 14H). Specifically, an increase in the mean absolute proportion of several immune cell types in mice receiving the ARG2 immune modulatory vaccine was found, including NK cells, and a tendency towards higher macrophage and monocyte numbers (FIG. 16). Higher average M1/M2 macrophage and CD8/Treg ratios (FIG. 14I and J) in mice receiving the ARG2-derived peptide vaccine indicated a more pro-inflammatory tumor microenvironment and suggested an immune modulatory capacity of ARG2-specific T cells induced upon vaccination.

TABLE 5
Overview of populations and subpopulations used in the
compact population scheme for the ImmuCC algorithm.
Compact Population Scheme
ImmuCC Algorithm

| Population | Sub-populations |
|---|---|
| B cells | B.Cells.Naive |
| | B.Cells.Memory |
| | Plasma.cells |
| CD8 T-cells | T.Cells.CD8.Naive |
| | T.Cells.CD8.Actived |
| | T.Cells.CD8.Memory |
| CD4 T-cells | T.Cells.CD4.Naive |
| | T.Cells.CD4.Memory |
| | T.Cells.CD4.Follicular |
| | Th1.Cells |
| | Th17.Cells |
| | Th2.Cells |
| Regulatory T-cells | — |
| Gamma-delta T-cells | — |
| NK cells | NK.Resting |
| | NK.Activated |
| DC cells | DC.Activated |
| | DC.Immature |
| Monocytes | — |
| Granulocytes | Eosinophil.Cells |
| | Mast.Cells |
| | Neutrophil.Cells |
| rarararon | MO.Macrophage |
| Macrophages | M1.Macrophage |
| | M2.Macrophage |

TABLE 6
Overview of populations and subpopulations used in the
extended population scheme for the ImmuCC algorithm.
Extended Population Scheme
ImmuCC Algorithm

| Population | Sub-populations |
|---|---|
| B cells | B.Cells.Naive |
| | B.Cells.Memory |
| | Plasma.cells |
| NK cells | NK.Resting |
| | NK.Activated |
| DC cells | DC.Activated |
| | DC.Immature |
| Granulocytes | Eosinophil.Cells |
| | Mast.Cells |
| | Neutrophil.Cells |
| T.Cells.CD8.Naive | — |
| T.Cells.CD8.Activated | — |
| T.Cells.CD8.Memory | — |
| T.Cells.CD4.Naive | — |
| T.Cells.CD4.Memory | — |
| T.Cells.CD4.Follicular | — |
| Treg.Cells | — |
| Th17.Cells | — |
| GammaDelta.T.cells | — |
| Mo.Macrophage | — |
| M1.Macrophage | — |
| M2.Macrophage | — |
| Monocytes | — |

The immune modulatory function of ARG2-specific T cells has thus been demonstrated in the murine Pan02 tumor model following ARG2-vaccination. Gene expression changes in Pan02 tumors from ARG2-vaccinated mice indicate the induction of an anti-tumor immune response in the form of increased immune cell infiltration and the establishment of a more pro-inflammatory microenvironment with higher M1/M2 macrophage and CD8/Treg ratios. The induction of a more immuno-permissive tumor microenvironment upon ARG2-vaccination could explain the significant inhibition of Pan02 tumor growth that was observed. Additionally, three other murine tumor models, namely MC38, B16-F10 and LL2, were used to validate the ability of ARG2-specific T cells to infiltrate the tumor bed. Overall, these results highlight the capability of ARG2-specific T cells to alter the immune landscape in favor of an anti-tumorigenic immune response.

### Sequences

Start pos and End pos indicate the positions within full length human Arginase 2 (SEQ ID NO: 19) unless otherwise indicated.

TABLE 1

| SEQ | | | | |
|---|---|---|---|---|
| ID | | | Start | End |
| NO | Sequence | Name | pos | pos |
| 1 | SLRGSLSRL | A201 | 2 | 10 |
| 2 | LLQTRVHSI | A2S02 | 10 | 18 |
| 3 | ILKKSVHSV | A2S03 | 18 | 26 |
| 4 | GLMKRLSSL | A2S04 | 53 | 61 |
| 5 | NLIVNPRSV | A2S05 | 84 | 92 |
| 6 | LANQELAEV | A2S06 | 94 | 102 |
| 7 | VTLGGDHSL | A2S07 | 114 | 122 |
| 8 | FLLRELQDKV | A2S08 | 166 | 175 |
| 9 | LLRELQDKV | A2S09 | 167 | 175 |
| 10 | ELQDKVPQL | A2S10 | 170 | 178 |
| 11 | IVYIGLRDV | A2S11 | 193 | 201 |
| 12 | KVMERTFDL | A2S12 | 229 | 237 |
| 13 | YIAEEIHNT | A2S13 | 278 | 286 |
| 14 | GLLSALDLV | A2S14 | 287 | 295 |
| 15 | LLSALDLVEV | A2S15 | 288 | 297 |
| 16 | GFSWIKPCISSA | “Hot spot” | 180 | 229 |
| | SIVYIGLRDVDP | | | |
| | PEHFILKNYDIQ | | | |
| | YFSMRDIDRLGI | | | |
| | QK | | | |
| 17 | SLRGSLSRLLQT | A2L2 | 2 | 34 |
| | RVHSILKKSVHS | | | |
| | VAVIGAPFS | | | |
| 18 | QIVKNPRSV | ARG1 65-73 | 65* | 73* |
| 19 | Full length | | | |
| | human ARG2 - | | | |
| | see below | | | |
| 20 | Full length | | | |
| | human ARG1 - | | | |
| | see below | | | |
| 21 | LSPPNIVYI | mARG2 188-196 | 188 | 196 |
| 22 | ISAKDIVYI | mARG1 169-177 | 169* | 177* |
| 23 | FSWVTPCISAKD | ARG1 (aa162-190) | 162* | 190* |
| | IVYIGLRDVDPG | | | |
| | EHYII | | | |
| 24 | FSWIKPCLSPPN | ARG2 (aa181-209) | 181 | 209 |
| | IVYIGLRDVEPP | | | |
| | EHFIL | | | |

*Positions with respect to full length human Arginase 1.

Full length human Arginase 2 (NP_001163.1)

(SEQ ID NO: 19)

MSLRGSLSRL LQTRVHSILK KSVHSVAVIG APFSQGQKRK
GVEHGPAAIR EAGLMKRLSS LGCHLKDFGD LSFTPVPKDD
LYNNLIVNPR SVGLANQELA EVVSRAVSDG YSCVTLGGDH
SLAIGTISGH ARHCPDLCVV WVDAHADINT PLTTSSGNLH
GQPVSFLLRE LQDKVPQLPG FSWIKPCISS ASIVYIGLRD
VDPPEHFILK NYDIQYFSMR DIDRLGIQKV MERTEDLLIG
KRQRPIHLSF DIDAFDPTLA PATGTPVVGG LTYREGMYIA
EEIHNTGLLS ALDLVEVNPQ LATSEEEAKT TANLAVDVIA
SSFGOTREGG HIVYDQLPTP SSPDESENQA RVRI
Region identified as a hot spot for
immunogenicity based on Arginase 1
homology is shown bold and underlined
(SEQ ID NO: 16). The transit peptide
boundary identified as the centre of
the A2L2 hotspot (SEQ ID NO: 17) is
shown bold and italic “KSV”.
Full length human Arginase 1 (NP_000036.2)

(SEQ ID NO: 20)

MSAKSRTIGI IGAPFSKGQP RGGVEEGPTV LRKAGLLEKL
KEQECDVKDY GDLPFADIPN DSPFQIVKNP RSVGKASEQL
AGKVAEVKKN GRISLVLGGD HSLAIGSISG HARVHPDLGV
IWVDAHTDIN TPLTTTSGNL HGQPVSFLLK ELKGKIPDVP
GFSWVTPCIS AKDIVYIGLR DVDPGEHYIL KTLGIKYFSM
TEVDRLGIGK VMEETLSYLL GRKKRPIHLS FDVDGLDPSE
TPATGTPVVG GLTYREGLYI TEEIYKTGLL SGLDIMEVNP
SLGKTPEEVT RTVNTAVAIT LACFGLAREG NHKPIDYLNP
PK
Region identified as a hot spot for
immunogenicity shown bold and underlined

1. A polypeptide which is an immunogenic fragment of human Arginase 2 (ARG2; SEQ ID NO: 19) that comprises or consists of a sequence of 9-19 consecutive amino acids of SEQ ID NO: 19 provided that said fragment does not comprise amino acids 2-34 or 180-229 of SEQ ID NO: 19.

2. The polypeptide of claim 1 which comprises or consists of a human leukocyte antigen (HLA) class I restricted epitope.

3. The polypeptide of any one of claim 1 or 2 which comprises or consists of a HLA-B8 restricted epitope.

4. The polypeptide of any one of the preceding claims which is capable of stimulating CD8⁺ T cells, optionally wherein the CD8⁺ positive T cells are cytotoxic T cells and/or are ARG2-specific.

5. The polypeptide of any one of the preceding claims which comprises or consists of the amino acid sequence: NLIVNPRSV (SEQ ID NO: 5).

6. The polypeptide of claim 1 which comprises or consists of a HLA class II restricted epitope.

7. The polypeptide of claim 1 or 6 which is capable of stimulating CD4⁺ T cells.

8. The polypeptide of any one of claim 1, 6 or 7 which comprises or consists of the amino acid sequence: GLLSALDLV (SEQ ID NO: 14).

9. The polypeptide of any one of the preceding claims, which has a maximum length of 9, 10, 11, 12, 13, 14, 15, 16, 17, 18 or 19 amino acids and/or in which the C terminal amino acid is replaced with the corresponding amide.

10. A polynucleotide encoding a polypeptide as defined in any one of the preceding claims, optionally comprised within a vector.

11. A composition comprising a polypeptide according to any one of claims 1-9 and/or a polynucleotide according to claim 10, and optionally an adjuvant.

12. The composition of claim 11 comprising:
a. at least one different polypeptide according to any one of claims 1-9, at least one different polynucleotide according to claim 10; and/or at least one pharmaceutically acceptable diluent, carrier or preservative; and/or
b. an adjuvant selected from the group consisting of bacterial DNA based adjuvants, oil/surfactant based adjuvants, viral dsRNA based adjuvants, imidazoquinolines, and a Montanide ISA adjuvant.

13. A method of treating or preventing a disease or condition in a subject, the method comprising administering to the subject the polypeptide of any one of claims 1 to 9, the polynucleotide of claim 10 and/or the composition of claim 11 or 12.

14. The method of claim 13 wherein:
a. the disease or condition is characterized at least in part by inappropriate or excessive immune suppressive function of ARG2, optionally wherein the excessive immune suppressive function of ARG2 is at least in part mediated by activated Treg cells expressing ARG2 and/or cancer-associated fibroblasts (CAFs) expressing ARG2; and/or
b. the disease or condition is cancer, optionally wherein the cancer is a melanoma (such as a malignant metastatic melanoma), chronic myeloid leukemia (CML), or pancreatic cancer.

15. The method of claim 13 or 14 wherein the disease or condition is cancer and optionally wherein the method further comprises the simultaneous or sequential administration of an additional cancer therapy to the subject, and optionally wherein the additional cancer therapy is an immune system checkpoint inhibitor, preferably an antibody, more preferably an anti-PD1 antibody.

16. A method of stimulating ARG2-specific T cells, the method comprising contacting the cells with the polypeptide of any one of claims 1 to 9 or the composition of claim 11 or 12, optionally wherein:
a. the cells are present in a sample taken from a healthy subject or from a cancer patient, optionally a tumor sample; and/or
b. the ARG-2 specific T cells are CD8⁺ positive T cells, preferably cytotoxic CD8⁺ T cells.
